(19) United States
(12) Patent Application Publication
(10) Pub. No.: US 2024/0299452 A1
(43) Pub. Date: Sep. 12, 2024

(54) Rapamycin and Cell Therapy

(71) Applicants: Innovative Cellular Therapeutics Holdings, Ltd., George Town (KY); Innovative Cellular Therapeutics, Inc., Rockville, MD (US)

(72) Inventors: Chengfei Pu, Shanghai (CN); Dongqi Chen, Shanghai (CN); Xiaogang Shen, Shanghai (CN); Le Tian, Rockville, MD (US)

(73) Assignees: Innovative Cellular Therapeutics Holdings, Ltd., George Town (KY); Innovative Cellular Therapeutics, Inc., Rockville, MD (US)

(21) Appl. No.: 18/441,683

(22) Filed: Feb. 14, 2024

Related U.S. Application Data
(60) Provisional application No. 63/485,151, filed on Feb. 15, 2023.

(51) Int. Cl. A61K 35/17; A61K 31/436; A61K 39/00; A61P 35/00; C07K 14/57; C07K 16/28

(52) U.S. Cl. CPC A61K 35/17; A61K 31/436; A61K 39/4612; A61K 39/4613; A61K 39/4631; A61K 39/464411; A61K 39/464441; A61P 35/00; C07K 14/57; C07K 16/2803; A61K 2239/13; A61K 2239/21; A61K 2239/58

(57) ABSTRACT

Embodiments of the present disclosure relate to compositions and methods of enhancing anti-tumor activities of modified cells, the method comprising: administering an effective amount of the modified cells to a subject having a solid tumor; and administering an effective amount of an agent to the subject, the agent comprising rapamycin, wherein the modified cells inhibit growth of the solid tumor in the subject, and wherein the anti-tumor activities in the subject are greater than those in a subject that is administered with an effective amount of modified cells but without the agent.

### CROSS REFERENCE TO RELATED APPLICATIONS

This application claims the benefit of U.S. Provisional Patent Application 63/485,151, filed on Feb. 15, 2023, which are hereby incorporated by reference in its entirety.

### BACKGROUND

Rapamycin, also known as Sirolimus, is a small molecule inhibitor that was initially discovered as an immunosuppressant in the soil of Easter Island. Today, it is widely used in transplant medicine to prevent rejection of transplanted organs.

T cell therapy is a type of immunotherapy that uses the patient's own T cells to target and destroy abnormal cells, such as cancer cells, infected cells, or self-reactive immune cells in autoimmune diseases. The process involves the extraction of T cells from the patient's blood, their genetic manipulation or activation in vitro, and their reinfusion back into the patient's bloodstream, where they can seek out and attack the target cells.

Rapamycin indeed reduces the risk of side effects associated with T cell therapy by slowing down T cell activation and proliferation. However, there is some evidence suggesting that rapamycin may also reduce the efficacy of T cell therapy by inhibiting the activation of T cells, which is critical for their function.

Further research is required to utilize rapamycin for mitigating the side effects induced by T cell therapy, while simultaneously preserving the crucial activation of T cells necessary for their optimal function.

### SUMMARY

Embodiments of the present disclosure relate to compositions and methods of enhancing anti-tumor activities of modified cells, the method comprising: administering an effective amount of the modified cells to a subject having a solid tumor; and administering an effective amount of an agent to the subject, the agent comprising rapamycin, wherein the modified cells inhibit growth of the solid tumor in the subject, and wherein the anti-tumor activities in the subject are greater than those in a subject that is administered with an effective amount of modified cells but without the agent.

This Summary is not intended to identify key features or essential features of the claimed subject matter, nor is it intended to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIGS. 1A-1C show constructs and expressions of CD19 CAR and PAP CAR in corresponding T cells.

FIG. 2 shows expansion of PAP CAR T cells in various culturing systems.

FIG. 3 shows cytokine release analysis of co-cultured cells with respect to PAP CAR and CD19 CAR.

FIG. 4 shows the protocol for the treatment using PAP CAR T cells.

FIG. 5 shows cytokines released by PAP CAR T cells in the body of a patient.

FIG. 6 shows a total prostate specific antigen (tPSA) assay in the peripheral blood (PB) of a patient.

FIG. 7 shows PET-CT scanning images of a patient one month after infusion of CAR T cells.

FIG. 8 shows PET-CT scanning images of the patient.

FIG. 9A shows dosages of CAR T cells infused to the patients.

FIG. 9B shows PSA level changes before and after CAR T cell infusion.

FIG. 10 depicts the fluctuation of PSA levels over time for Group 1.

FIG. 11 illustrates the variation of PSA levels over time for Group 2.

FIG. 12 shows the scheme of administration of rapamycin to Patient 04 after the infusion of CAR T cells.

FIG. 13 shows T regulator cells (Tregs) were reduced in tumor tissue.

FIG. 14 shows the impact of rapamycin on CAR T cell activation and Exhaustion Markers.

FIG. 15 shows the distribution of T-cell phenotypes under varied treatment conditions.

FIG. 16 shows the effects of rapamycin on cytokine production by CAR T cells.

FIG. 17 shows rapamycin's influence on CAR T cell proliferation.

FIG. 18 shows rapamycin upregulates IFNγ sensitivity in prostate cancer cells.

FIG. 19 summarizes the findings related to rapamycin use in cell therapies.

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies, monoclonal antibodies, and Fv, Fab, Fab′ and F(ab′)2 and fragments, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab′, F(ab′)2, and Fv fragments; diabodies; linear antibodies; single-chain antibody molecules; and multi-specific antibodies formed from antibody fragments.

The term “Fv” refers to the minimum antibody fragment which contains a complete antigen-recognition and-binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody, or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “autoantigen” or “self-antigen” refers to an antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be related or unrelated to the recipient subject, but the donor subject has immune system markers which are similar to the recipient subject.

The term “xenogeneic” is used to describe a graft derived from a subject of a different species. As an example, the donor subject is from a different species than a recipient subject and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal Cancer |
| MUC12 | colorectal Cancer |
| GUCY2C (GCC) | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL-13Rα2 (IL-13 | Glioma |
| receptor alpha 2) | |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinomas |
| EGFRvIII | Glioma-Glioblastoma |
| EGFR | Glioma-NSCL cancer |
| tMUC 1 | Cholangiocarcinoma, Pancreatic cancer, |
| PSCA | pancreas, stomach, or prostate cancer |

The term “tumor associated antigens (TAAs)” as used herein refers to antigens selectively expressed or overexpressed by malignant cells in a tissue with a tumor as compared with a corresponding normal tissue. The tumor associated antigens include various groups such as tumor specific antigens, oncogetal antigens, oncogene products, organ lineage antigens, viral antigens, etc. For example, oncogene and suppressor gene products, such as nonmutated HER-2/neu and p53, are analogous to oncofetal antigens in that they can be overexpressed in tumors and may be expressed in some fetal tissues. Additional examples of TAAs include Fibroblast activation protein-α (FAP), HER2, MART-1, MUC1, tyrosinase, MAGE, mammaglobin-A, and NY-ESO-1.

For example, FAP is a type II integral serine protease that is specifically expressed by activated fibroblasts. Cancer-associated fibroblasts (CAFs) in the tumor stroma have an abundant and stable expression of FAP, which plays an important role in promoting tumor growth, invasion, metastasis, and immunosuppression. For example, in females with a high incidence of breast cancer, CAFs account for 50-70% of the cells in the tumor's microenvironment. CAF overexpression of FAP promotes tumor development and metastasis by influencing extracellular matrix remodeling, intracellular signaling, angiogenesis, epithelial-to-mesenchymal transition, and immunosuppression.

The term “tumor specific antigens (TSAs)” as used herein refers to antigens that are uniquely expressed in tumors, such as point-mutated ras oncogenes, p53 mutations, anti-idiotype antibodies (Abs), and products of ribonucleic acid (RNA) splice variants, and gene translocations. Another example of TSA is tumor form of human MUC1 (tMUC1).

For example, MUC1 is one of the epithelial mucin family of molecules. MUC1 is a transmembrane mucin glycoprotein that is normally expressed on all glandular epithelial cells of the major organs. In normal cells, MUC1 is only expressed on the apical surface and is heavily glycosylated with its core proteins sequestered by the carbohydrates. As cells transform to a malignant phenotype, expression of MUC1 increases several folds, and the expression is no longer restricted to the apical surface, but it is found all around the cell surface and in the cytoplasm. In addition, the glycosylation of tumor associated MUC1 (tMUC1) is aberrant, with greater exposure of the peptide core than is found on MUC1 expressed in normal tissues.

The term “organ lineage antigen (OLA)” as used herein refers to an antigen expressed in a tumor of a given type and the normal organ from which the tumor is derived. Examples of organ lineage antigen include prostate-specific antigen (PSA) and the melanocyte antigens, such as CD19, BCMA, CD20, CD22, GCC, PAP, MSLN, and ALPP. Organ lineage antigens can serve as targets for immunotherapy if the normal organ in which they are expressed is not essential, such as the prostate, breast, or melanocyte. As used herein, an organ refers to an integrated group of cells with a common structure, an intercellular material, and/or a function.

For example, guanylyl cyclase 2C (GUCY2C or GCC) is principally expressed in intestinal epithelial cells. GUCY2C is the receptor for diarrheagenic bacterial enterotoxins and the gut paracrine hormones, guanylin, and uroguanylin. These ligands regulate water and electrolyte transport in the intestinal and renal epithelia and are ultimately responsible for acute secretory diarrhea.

Throughout this specification, unless the context requires otherwise, the words “comprise,” “includes” and “including” will be understood to imply the inclusion of a stated step or element (ingredient or component) or group of steps or elements (ingredients or components) but not the exclusion of any other step or element or group of steps or elements.

The phrase “consisting of” is meant to include, and is limited to, whatever follows the phrase “consisting of.” Thus, the phrase “consisting of” indicates that the listed elements or steps are required or mandatory and that no other elements may be present.

The phrase “consisting essentially of” is meant to include any element listed after the phrase and can include other elements or steps that do not interfere with or contribute to the activity or action specified in the disclosure for the listed elements or steps. Thus, the phrase “consisting essentially of” indicates that the listed elements or steps are required or mandatory, but that other elements or steps are optional and may or may not be present depending upon whether or not they affect the activity or action of the listed elements or steps. In embodiments, those elements or steps that do not affect an embodiment are those elements or steps that do not alter the embodiment's ability in a statistically significant manner to perform a function in vitro or in vivo, such as killing cancer cells in vitro or in vivo.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “co-stimulatory signaling region”, “co-stimulatory domain”, and “co-stimulation domain” are used interchangeably to refer to one or more additional stimulatory domain in addition to a stimulatory or signaling domain such as CD3 zeta. The terms “stimulatory” or “signaling” domain (or region) are also used interchangeably, when referring, for example, to CD3 zeta. In embodiments, the co-stimulatory signaling domain and the signaling domain can be on the same molecule or different molecules in the same cell.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for the synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses (AAV)) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially free from components that normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein, and refer to any animal, such as a mammal, for example, a human or any living organism amenable to the methods described herein. In embodiments, the patient, subject, or individual is a human or mammal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder. Accordingly, the subject can also be in need of prevention of a disease condition or disorder. In embodiments, the disease is cancer.

The term “polynucleotide” or “nucleic acid” refers to mRNA, RNA, CRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In embodiments, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

“NFAT promoter” refers to one or more NFAT binding sites or motifs linked to a minimal promoter of any gene expressed by T cells. In embodiments, the minimal promoter of a gene expressed by T cells is a minimal human IL-12 promoter. Nuclear factor of activated T cells (NFAT) are transcription factors. Examples of NFAT transcription factors include NFAT1, NFAT2, NFAT3, NFAT4, and NFAT5. These transcription factors bind NFAT binding sites or motifs in the NFAT promoter. The NFAT promoter (or a functional portion or functional variant thereof) can comprise any number of binding motifs, e.g., at least two, at least three, at least four, at least five, or at least six, at least seven, at least eight, at least nine, at least ten, at least eleven, or up to twelve binding motifs. In embodiments, the NFAT promoter comprises six NFAT binding motifs.

The NFAT promoter (or a functional portion or functional variant thereof) is operatively associated with the nucleotide sequence encoding IL-12 (or a functional portion or functional variant thereof). “Operatively associated with” means that the nucleotide sequence encoding IL-12 (or a functional portion or functional variant thereof) is transcribed into IL-12 mRNA when the NFAT protein binds to the NFAT promoter sequence (or a functional portion or functional variant thereof). Without being bound to a particular theory, it is believed that NFAT is regulated by a calcium signaling pathway. In particular, it is believed that TCR stimulation (by, e.g., an antigen) and/or stimulation of the calcium signaling pathway of the cell (by, e.g., PMA/lonomycin) increases intracellular calcium concentration and activates calcium channels. It is believed that the NFAT protein is then dephosporylated by calmoduin and translocates to the nucleus where it binds the NFAT promoter sequence (or a functional portion or functional variant thereof) and activates downstream gene expression. By providing an NFAT promoter (or a functional portion or functional variant thereof) that is operatively associated with the nucleotide sequence encoding IL-12 (or a functional portion or functional variant thereof), the nucleic acids described herein advantageously make it possible to express IL-12 (or a functional portion or functional variant thereof) only when the host cell including the nucleic acid is stimulated by, e.g., PMA/lonomycin and/or an antigen. More information can be found at U.S. Pat. No. 8,556,882, which is incorporated by the reference.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains can be “engineered” to bind to a predetermined nucleotide sequence, for example via engineering (altering one or more amino acids) of the recognition helix region of a naturally occurring zinc finger protein. Further, a Zinc finger binding domain may be fused a DNA-cleavage domain to form a Zinc finger nuclease (ZFN) targeting a specific desired DNA sequence. For example, a pair of ZFNs (e.g., a ZFN-left arm and a ZFN-right arm) may be engineered to target and cause modifications of specific desired DNA sequences (e.g., TRAC genes).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In embodiments, fusion polypeptides are used for targeted double-stranded DNA cleavage.

A “target site” or “target sequence” is a nucleic acid sequence that defines a portion of a nucleic acid to which a binding molecule will bind, provided sufficient conditions for binding exist. For example, the sequence 5′ GAATTC 3′ is a target site for the Eco RI restriction endonuclease.

A “fusion” molecule is a molecule in which two or more subunit molecules are linked, preferably covalently. The subunit molecules can be the same chemical type of molecule or can be different chemical types of molecules. Examples of the first type of fusion molecule include, but are not limited to, fusion proteins (for example, a fusion between a ZFP DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage, and polypeptide ligation can also be involved in the expression of the protein in a cell. Methods for polynucleotide and polypeptide delivery to cells are presented elsewhere in this disclosure.

“Modulation” of gene expression refers to a change in the activity of a gene. Modulation of expression can include but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures. CD3 zeta is not the only suitable primary signaling domain for a CAR construct with respect to the primary response. For example, back in 1993, both CD3 zeta and FcRy were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T cell receptors” PNAS, 1993 Jan. 15; 90(2):720-4, showed that two CAR constructs in which an scFv was fused to “either the FcR gamma chain or the CD3 complex chain” triggered T cell activation and target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al. (,“A Chimeric Antigen Receptor (CARs) Based Upon a Killer Immunoglobulin-Like Receptor (KIR) Triggers Robust Cytotoxic Activity in Solid Tumors” Molecular Therapy, vol. 22, no. Suppl. 1, May 2014, page S57) tested a CAR molecule in which an scFv was fused to “the transmembrane and cytoplasmic domain of a killer immunoglobulin-like receptor (KIR). Wang et al. reported that, “a KIR-based CAR targeting mesothelin (SS 1-KIR) triggers antigen-specific cytotoxic activity and cytokine production that is comparable to CD3˜-based CARs.” A second publication from the same group, Wang et al. (“Generation of Potent T-cell Immunotherapy for Cancer Using DAP12-Based, Multichain, Chimeric Immunoreceptors” Cancer Immunol Res. 2015 July; 3(7):815-26) showed that a CAR molecule in which “a single-chain variable fragment for antigen recognition was fused to the transmembrane and cytoplasmic domains of KIR2DS2, a stimulatory killer immunoglobulin-like receptor (KIR)” functioned both in vitro and in vivo “when introduced into human T cells with DAP12, an immunotyrosine-based activation motifs-containing adaptor.”

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen presenting cell (e.g., an APC, a dendritic cell, a B-cell, and the like.) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

The term “vector” refers to a polynucleotide that comprises an isolated nucleic acid and which can be used to deliver the isolated nucleic acid to the interior of a cell. The cell can be an in vitro cell or a in vivo cell in a subject. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

In embodiments, a polynucleotide encoding the antigen binding molecule and/or therapeutic agent(s) can be used to implement techniques described herein. The method or use includes: providing a viral particle (e.g., AAV, lentivirus or their variants) comprising a vector genome, the vector genome comprising the polynucleotide, wherein the polynucleotide is operably linked to an expression control element conferring transcription of the polynucleotide; and administering an amount of the viral particle to the subject such that the polynucleotide is expressed in the subject. In embodiments, the AAV preparation may include AAV vector particles, empty capsids and host cell impurities, thereby providing an AAV product substantially free of AAV empty capsids. More information of the administration and preparation of the viral particle may be found at the U.S. Pat. No. 9,840,719 and Milani et al., Sci. Transl. Med. 11, eaav7325 (2019) 22 May 2019, which are incorporated herein by reference. In embodiments, the polynucleotide may integrate into the genome of the modified cell and the progeny of the modified cell will also express the polynucleotide, resulting in a stably transfected modified cell. In embodiments, the modified cell expresses the polynucleotide encoding the CAR but the polynucleotide does not integrate into the genome of the modified cell such that the modified cell expresses the transiently transfected polynucleotide for a finite period of time (e.g., several days), after which the polynucleotide is lost through cell division or other factors. For example, the polynucleotide is present in the modified cell in a recombinant DNA construct, in an mRNA, or in a viral vector, and/or the polynucleotide is an mRNA, which is not integrated into the genome of the modified cell.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The T cell response in a subject refers to cell-mediated immunity associated with a helper, killer, regulatory, and other types of T cells. For example, T cell response may include activities such as assistance to other white blood cells in immunologic processes and identifying and destroying virus-infected cells and tumor cells. T cell response in the subject may be measured via various indicators such as the number of virus-infected cells and/or tumor cells that T cells kill, an amount of cytokines that T cells release, for example, in co-culturing with virus-infected cells and/or tumor cells, a level of proliferation of T cells in the subject, a phenotype change of T cells (e.g., changes to memory T cells), and the longevity or lifespan of T cells in the subject.

In embodiments, in vitro killing assay may be performed by measuring the killing efficacy of CAR T cells by co-culturing CAR T cells with antigen-positive cells. CAR T cells may be considered to have killing effect on the corresponding antigen-positive cells by showing a decrease in the number of corresponding antigen-positive cells co-cultured with CAR T cells and an increase in the release of cytokines such as IFN-γ, TNF-α, and the like, as compared to control cells that do not express the corresponding antigen. Further, in vivo antitumor activity of the CAR T cells may be tested. For example, xenograft models can be established using the antigens described herein in immunodeficient mice. Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2, -247-250 (2007)). To evaluate the anti-tumor activity of CAR T cells in vivo, immunodeficient mice bearing tumor xenografts were evaluated for CAR T cell anti-tumor activity, for example, a decrease in mouse tumors and/or mouse blood cytokines, such as IFN-γ, TNF-α, and the like.

The term “chimeric antigen receptor” or alternatively a “CAR” refers to a recombinant polypeptide construct comprising at least an extracellular antigen binding domain, a transmembrane domain, and an intracellular signaling domain (e.g., cytoplasmic domain). In embodiments, the domains in the CAR polypeptide are on the same polypeptide chain (e.g., comprising a chimeric fusion protein). In embodiments, the domains of the CAR polypeptide are not on the same molecule, e.g. not contiguous with each other, or are on different polypeptide chains.

In embodiments, the intracellular signaling domain may include a functional signaling domain derived from a stimulatory molecule and/or a co-stimulatory molecule as described herein. In embodiments, the intracellular signaling domain includes a functional signaling domain derived from a primary signaling domain (e.g., a primary signaling domain of CD3-zeta). In embodiments, the intracellular signaling domain further includes one or more functional signaling domains derived from at least one co-stimulatory molecule. The co-stimulatory signaling region refers to a portion of the CAR including the intracellular domain of a co-stimulatory molecule. Co-stimulatory molecules can include cell surface molecules for inducing an efficient response from the lymphocytes (in response to an antigen).

Between the extracellular domain and the transmembrane domain of the CAR, there can be incorporated a spacer domain. As used herein, the term “spacer domain” generally means any oligo- or polypeptide that functions to link the transmembrane domain to the extracellular domain and/or the cytoplasmic domain in the polypeptide chain. A spacer domain may include up to 300 amino acids, 10 to 100 amino acids, or 25 to 50 amino acids.

The extracellular domain of a CAR may include an antigen binding domain (e.g., a scFv, a single domain antibody, or TCR, such as a TCR alpha binding domain or a TCR beta binding domain), that targets a specific tumor marker (e.g., a tumor antigen). Tumor antigens are proteins that are produced by tumor cells that elicit an immune response, particularly T cell mediated immune responses. Tumor antigens are well known in the art and include, for example, a glioma-associated antigen, carcinoembryonic antigen (CEA), B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. For example, when the antigen that the CAR binds is CD19, the CAR thereof is referred to as CD19 CAR (19CAR, CD19CAR, CD19 CAR, or CD19-CAR), which is a CAR molecule that includes an antigen binding domain that binds CD19.

In embodiments, the extracellular ligand-binding domain comprises a scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence, which bridges approximately 3.5 nm between the carboxy terminus of one variable region and the amino terminus of the other variable region. Linkers of other sequences have been designed and used (Bird et al., 1988, supra). In general, linkers can be short, flexible polypeptides comprising about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor a 2, IL-11 receptor a, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by a tumor. In embodiments, the binding element of the CAR includes any antigen binding moiety that when bound to its cognate antigen, affects a tumor cell such that the tumor cell fails to grow, decrease in size, or dies.

The CAR can be a bispecific CAR. For example, the two antigen binding domains are on the same CAR (a bispecific CAR or tandem CAR (tanCAR)), on different CAR molecules, or on a CAR and T cell receptor (TCR). A single CAR can include two different antigen binding domains, or the two different antigen binding domains are each on a separate CAR. The CAR can have more than two antigen binding domains, for example, a multispecific CAR. The antigen binding domains of the multispecific CAR can be on the same CAR or on separate CAR, such as one antigen binding domain on each CAR.

In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.

In embodiments, the intracellular domain comprises a CD3 zeta signaling domain. Embodiments relate to a vector comprising the isolated nucleic acid sequence described herein. Embodiments relate to an isolated cell comprising the isolated nucleic acid sequence described herein.

The cells, including CAR cells and modified cells, described herein can be derived from a stem cell. The stem cells may be adult stem cells, embryonic stem cells, or non-human stem cells, cord blood stem cells, progenitor cells, bone marrow stem cells, induced pluripotent stem cells, totipotent stem cells, or hematopoietic stem cells. The cells can also be a dendritic cell, a NK-cell, a B-cell, or a T cell selected from the group consisting of inflammatory T lymphocytes, cytotoxic T lymphocytes, regulatory T lymphocytes, and helper T lymphocytes. In embodiments, the cells can be derived from the group consisting of CD4+T-lymphocytes and CD8+T-lymphocytes. Prior to expansion and genetic modification of the cells described herein, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T cell lines available and known to those skilled in the art, can be used. In embodiments, the cells may be derived from a healthy donor, from a patient diagnosed with cancer, or from a patient diagnosed with an infection. In embodiments, the cells are part of a mixed population of cells which present different phenotypic characteristics.

A population of cells refers to a group of two or more cells. The cells of the population could be the same, such that the population is a homogenous population of cells. The cells of the population could be different, such that the population is a mixed population or a heterogeneous population of cells. For example, a mixed population of cells could include modified cells comprising a first CAR and cells comprising a second CAR, wherein the first CAR and the second CAR bind different antigens.

The term “stem cell” refers to any type of cell which has the capacity for self-renewal and the ability to differentiate into other kind(s) of cell. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cells may be distinguished on the basis of their origin and/or on the extent of their capacity for differentiation into other types of cell. Stem cells can include embryonic stem (ES) cells (i.e., pluripotent stem cells), somatic stem cells, induced pluripotent stem cells, and any other types of stem cells.

Pluripotent embryonic stem cells can be found in the inner cell mass of a blastocyst and have high innate capacity for differentiation. For example, pluripotent embryonic stem cells have the potential to form any type of cell in the body. When grown in vitro for long periods of time, ES cells maintain pluripotency, and progeny cells retain the potential for multilineage differentiation.

Somatic stem cells can include fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells; they apparently differentiate into only a limited number of different types of cells and have been described as multipotent. “Tissue-specific” stem cells normally give rise to only one type of cell. For example, embryonic stem cells can differentiate into blood stem cells (e.g., Hematopoietic stem cells (HSCs)), which can further differentiate into various blood cells (e.g., red blood cells, platelets, white blood cells, etc.).

Induced pluripotent stem cells (iPS cells or iPSCs) can include a type of pluripotent stem cell artificially derived from a non-pluripotent cell (e.g., an adult somatic cell) by inducing expression of specific genes. Induced pluripotent stem cells are similar to naturally occurring pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be isolated from adult stomach, liver, skin cells, and blood cells.

In embodiments, the CAR cells, the modified cell, or the cell is a T cell, a NK cell, a macrophage or a dendritic cell. For example, the CAR cells, the modified cell, or the cell is a T cell.

T cells, or T lymphocytes, are a type of white blood cell of the immune system. There are various types of T cells including T helper (TH) cells, cytotoxic T (TC) cells (T killer cells, killer T cells), natural killer T (NKT) cells, memory T (Tm) cells, regulatory T (Treg) cells, and gamma delta T (γδ T) cells.

T helper (TH) cells assist other lymphocytes, for example, activating cytotoxic T cells and macrophages and maturation of B cells into plasma cells and memory B cells. These T helper cells express CD4 glycoprotein on their surface and are also known as CD4+ T cells. Once activated, these T cells divide rapidly and secrete cytokines.

Cytotoxic T (TC) cells destroy virus-infected cells and tumor cells and are also involved in transplant rejection. They express CD8 protein on their surface. Cytotoxic T cell release cytokines.

Natural Killer T (NKT) cells are different from natural killer cells. NKT cells recognize glycolipid antigens presented by CD1d. Once activated, NKT cells produce cytokine and release cell killing molecules.

Memory T (Tm) cells are long-lived and can expand to large number of effector T cells upon re-exposure to their cognate antigen. Tm cells provide the immune system with memory against previously encountered pathogens. There are various subtypes of Tm cells including central memory T (TCM) cells, effector memory T (TEM) cells, tissue resident memory T (TRM) cells, and virtual memory T cells. Tm cells are either CD4+ or CD8+ and usually CD45RO.

Regulatory T (Treg) cells shut down T cell mediated immunity at the end of an immune reaction and suppress autoreactive T cells that escaped the process of negative selection in the thymus. Subsets of Treg cells include thymic Treg and peripherally derived Treg. Both subsets of Treg require the expression of the transcription factor FOXP3.

Gamma delta T (γδ T) cells are a subset of T cells that possess a γδ T cell receptor (TCR) on the cell surface, as most T cells express the αβ TCR chains. γδ T cells are less common in human and mice and are mainly found in the gut mucosa, skin, lung, and uterus. They are involved in the initiation and propagation of immune responses.

In embodiments, the antigen binding molecule is a T Cell Receptor (TCR). In embodiments, the TCR is modified TCR. In embodiments, the TCR is derived from spontaneously occurring tumor-specific T cells in patients. In embodiments, the TCR binds a tumor antigen. In embodiments, the tumor antigen comprises CEA, gp100, MART-1, p53, MAGE-A3, or NY-ESO-1. In embodiments, the TCR comprises TCRγ and TCRδ chains or TCRα and TCRβ chains.

In embodiments, a T cell clone that expresses a TCR with high affinity for the target antigen may be isolated. In embodiments, tumor-infiltrating lymphocytes (TILs) or peripheral blood mononuclear cells (PBMCs) may be cultured in the presence of antigen-presenting cells (APCs) pulsed with a peptide representing an epitope known to elicit a dominant T cell response when presented in the context of a defined HLA allele. High-affinity clones may be then selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRβ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle (e.g., a gammaretrovirus or lentivirus) may be then generated and tested for functionality (antigen specificity and functional avidity) and used to produce a clinical lot of the vector. An aliquot of the final product is then used to transduce the target T cell population (generally purified from patient PBMCs), which is expanded before infusion into the subject.

In embodiments, the APCs include dendritic cells, macrophages, Langerhans cells and B cells, or T cells.

In embodiments, the binding element of the CAR may include any antigen binding moiety that when bound to its cognate antigen, affects a tumor cell for example, it kills the tumor cell, inhibits the growth of the tumor cell, or promotes death of the tumor cell.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

The embodiments of the present disclosure further relate to vectors in which a nucleic acid described herein is inserted. Vectors can be derived from retroviruses such as the lentivirus that are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for deliverying nucleic acids into a cell, for example, electroporation, gene gun, sonoporation, magnetofection, and the use of oligonucleotides, lipoplexes, dendrimers, and inorganic nanoparticles.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration into eukaryotes. Typical cloning vectors contain transcription and translation terminators, initiation sequences, and promoters useful for regulation of the expression of the desired nucleic acid sequence.

Additional information related to expression of synthetic nucleic acids encoding CARs and gene transfer into mammalian cells is provided in U.S. Pat. No. 8,906,682, incorporated by reference in its entirety.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, “therapeutic amount”, or “effective amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T cells described herein may be administered at a dosage of 104 to 109cells/kg body weight, preferably 105 to 106 cells/kg body weight, including all integer values within those ranges. T cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art by monitoring the patient for signs of disease and adjusting the treatment accordingly. In embodiments, activated T cells are administered to a subject and then subsequently blood is redrawn (or have apheresis performed). T cells are collected, expanded, and reinfused into the subject. This process can be carried out multiple times every few weeks. In embodiments, T cells can be activated from blood draws of from 10 cc to 400 cc. In embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, certain populations of T cells can be selected.

The administration of the pharmaceutical compositions described herein can be carried out in any convenient manner, including by aerosol inhalation, injection, ingestion, transfusion, implantation or transplantation. The pharmaceutical compositions described herein can be administered to a patient subcutaneously, intradermally, intratumorally, intranodally, intramedullary, intramuscularly, intravenously (i. v.), or intraperitoneally. In embodiments, the T cell compositions of the present disclosure are administered to a patient by intradermal or subcutaneous injection. In embodiments, the T cell compositions of the present disclosure are administered by i.v. injection. The compositions of T cells may be injected directly into a tumor, lymph node, or site of infection. In embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In embodiments, the cell compositions of the present disclosure are administered following B-cell ablative therapy such as agents that react with CD20, e.g., Rituxan®. For example, subjects may undergo standard treatment with high dose chemotherapy followed by peripheral blood stem cell transplantation. In embodiments, following the transplant, subjects receive an infusion of the expanded immune cells of the present disclosure. In embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician depending on various factors.

Additional information on the methods of cancer treatment using engineered or modified T cells is provided in U.S. Pat. No. 8,906,682, incorporated by reference in its entirety.

In embodiments, the population of cells described herein is used in autologous CAR T cell therapy. In embodiments, the CAR T cell therapy is allogenic CAR T cell therapy, TCR T cell therapy, and NK cell therapy.

Embodiments relate to an in vitro method for preparing modified cells. The method may include obtaining a sample of cells from the subject. For example, the sample may include T cells or T cell progenitors. The method may further include transfecting the cells with a DNA encoding at least a CAR, culturing the population of CAR cells ex vivo in a medium that selectively enhances proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

As used herein, the term “gene fusion” refers to the fusion of at least a portion of a gene to at least a portion of an additional gene. The gene fusion need not include entire genes or exons of genes. In some instances, gene fusion is associated with alternations in cancer. A gene fusion product refers to a chimeric genomic DNA, a chimeric messenger RNA, a truncated protein or a chimeric protein resulting from a gene fusion. The gene fusion product may be detected by various methods described in U.S. Pat. No. 9,938,582, which is incorporated as a reference herein. A “gene fusion antigen” refers to a truncated protein or a chimeric protein that results from a gene fusion. In embodiments, an epitope of a gene fusion antigen may include a part of the gene fusion antigen or an immunogenic part of another antigen caused by the gene fusion. In embodiments, the gene fusion antigen interacts with, or is part of, cell membranes.

In embodiments, detection of mRNA and protein expression levels of the target molecules (e.g., CARs and cytokines) in human cells may be performed using experimental methods such as qPCR and FACS. Further, target molecules specifically expressed in the corresponding tumor cells with very low expression or undetectable expression in normal tissue cells may be identified.

In embodiments, In Vitro Killer Assay as well as killing experiment of CAR T Cells Co-Cultured with Antigen-Positive Cells can be performed. CAR T cells can exhibit a killing effect on the corresponding antigen-positive cells, a decrease in the number of corresponding antigen-positive cells co-cultured with CAR T cells, and an increase in the release of IFN-γ, TNF-α, etc. as compared to control cells that did not express the corresponding antigen.

In embodiments, In Vivo Killer Assay can be performed. For example, mice may be transplanted with corresponding antigen tumor cells, and tumorigenic, transfusion of CAR T cells, and a decrease in mouse tumors and mouse blood IFN-γ, TNF-α, and other signals can be defected.

Embodiments relate to a method of eliciting and/or enhancing T cell response in a subject having a solid tumor or treating a solid tumor in the subject, the method comprising administering an effective amount of T cells comprising the CAR described herein. In embodiments, the intracellular domain of the CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the intracellular domain comprises a CD3 zeta signaling domain.

Embodiments relate to a vector comprising the isolated nucleic acid described herein.

Embodiments relate to an isolated cell comprising the isolated nucleic acid sequence described herein. Embodiments relate to a composition comprising a population of T cells comprising the CAR described herein. Embodiments relate to a CAR encoded by the isolated nucleic acid sequence described herein. Embodiments relate to a method of eliciting and/or enhancing T cell response in a subject or treating a tumor of the subject, the method comprising: administering an effective amount of T cell comprising the CAR described herein.

In embodiments, the CAR molecules described herein comprise one or more complementarity-determining regions (CDRs) for binding an antigen of interest. CDRs are part of the variable domains in immunoglobulins and T cell receptors for binding a specific antigen. There are three CDRs for each variable domain. Since there is a variable heavy domain and a variable light domain, there are six CDRs for binding an antigen. Further since an antibody has two heavy chains and two light chains, an antibody can have twelve CDRs altogether for binding antigens.

In embodiments, the modified cells described herein includes a CAR molecule comprising at least two different antigen binding domains. The CAR molecule can be a bispecific CAR molecule. For example, the two antigen binding domains can be on the same CAR molecule, on different CAR molecules, or on a CAR molecule and T cell receptor (TCR). A single CAR can include at least two different antigen binding domains, or the two different antigen binding domains are each on a separate CAR molecule. The at least two different antigen binding domains can be on the same CAR molecule or different CAR molecules, but in the same modified cell. Moreover, the at least two different antigen binding domains can be on a CAR molecule and a T cell receptor in the same modified cell. In embodiments, the bispecific CAR molecule can include a binding domain binding an antigen of WBC (e.g., CD19) and a binding domain binding a solid tumor antigen. In embodiments, the bispecific CAR molecule may include two binding domains binding two different solid tumor antigens.

In embodiments, the at least two different antigen binding domains are on different CAR molecules which are expressed by different modified cells. Further, the one or more different antigen binding domains are on a CAR molecule and a T cell receptor, which are expressed by different modified cells.

While CAR T cell therapy provides vigorous antitumor activities against blood tumors, CAR T therapy alone, at least for certain solid tumor types, may not be enough to overcome the tumor microenvironment, inhibit tumor growth, and eventually treat cancer patients. Therapeutic agents such as cytokine may enhance cell therapy when immune cells are modified to express a therapeutic agent in the body of patients. However, the expression of therapeutic agents must be regulated to avoid potential toxicity caused by the therapeutic agents.

Rapamycin is a drug that can enhance the effectiveness and longevity of T cell therapy. It works by inhibiting the mTOR signaling pathway, which regulates T cell metabolism, proliferation, and differentiation. By suppressing mTOR, rapamycin can promote the survival and memory formation of T cells, as well as their ability to infiltrate and function in tumor tissues. Rapamycin can also modulate the balance between effector and regulatory T cells, which can help to prevent immune overactivation and side effects. Overall, rapamycin and its derivatives have shown promise as adjuvants to T cell therapy in preclinical and clinical studies.

In T cell therapy, the extracted T cells are genetically modified to express a chimeric antigen receptor (CAR) or a T cell receptor (TCR) that recognizes a specific target on cancer cells. This enhances the ability of T cells to specifically recognize and kill cancer cells. However, this process also activates the immune system, leading to rapid expansion and activation of T cells, which can result in serious side effects such as cytokine release syndrome (CRS) and neurological toxicity.

Rapamycin is used in T cell therapy to control the activation and proliferation of T cells. It works by binding to the protein complex mTOR, which is a critical regulator of cellular metabolism, growth and proliferation. By inhibiting mTOR, Rapamycin slows down T cell activation and expansion, reducing the risk of side effects and improving the safety of T cell therapy. While rapamycin has shown promise in enhancing the efficacy and longevity of T cell therapy, some studies have suggested that it may also inhibit the activation of T cells, which could compromise their function and ultimately reduce the therapy's effectiveness.

T cell activation involves a complex signaling cascade that is regulated by multiple pathways, including the mTOR pathway that rapamycin targets. By inhibiting mTOR, rapamycin can interfere with the metabolic and transcriptional changes that occur during T cell activation, such as upregulation of cytokines, chemokines, and adhesion molecules that promote T cell proliferation, differentiation, and migration to the site of infection or tumor. In addition, rapamycin can induce the generation of regulatory T cells, which can suppress the activity of effector T cells and reduce their ability to attack target cells.

There have been several preclinical and clinical studies that have suggested that rapamycin can compromise the function of T cells and reduce the effectiveness of T cell therapy. In a mouse model of melanoma, treatment with rapamycin reduced the infiltration and activation of adoptively transferred T cells in the tumor microenvironment, resulting in decreased tumor regression and survival compared to untreated mice. In a phase I clinical trial of adoptive T cell therapy for metastatic melanoma, patients who received high-dose rapamycin as a conditioning regimen prior to T cell infusion had a lower response rate and shorter progression-free survival than patients who received a low-dose or no rapamycin conditioning. In a study of CAR-T cell therapy for acute lymphoblastic leukemia, rapamycin was found to impair the expansion and function of CAR-T cells in vitro, as well as reduce their antitumor activity in vivo, by inhibiting their cytokine production and cytotoxicity. In a study of T cell therapy for chronic viral infections, such as HIV and hepatitis B virus, rapamycin was found to induce the generation of regulatory T cells that suppress the activity of antiviral T cells and impair the clearance of the viral infection.

Therefore, the optimal use of rapamycin in T cell therapy would require a careful balance between enhancing the survival and memory formation of T cells, while minimizing the inhibition of their activation and effector functions. This may involve optimizing the dose, duration, and timing of rapamycin treatment, as well as combining rapamycin with other immunomodulatory agents that can synergize with or counteract its effects.

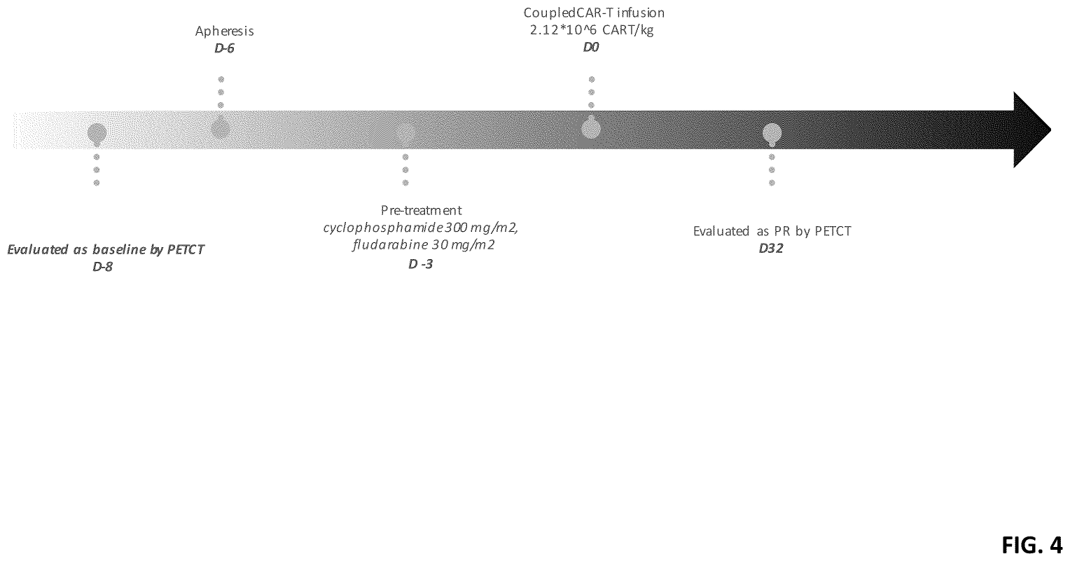

Rapamycin is a commonly used drug that has been shown to decrease the activity of T cells, but it can also increase the number of Treg cells, limiting its usefulness in CAR T cell therapy. Interestingly, as shown in Example, CoupledCAR™ can reduce the number of Treg cells in the tumor tissues. By using rapamycin to decrease the activity of T cells and TA to reduce the number of Treg cells, CAR T cells can be more effective at targeting and killing cancer cells. In addition, this combination has been shown to decrease the severity of adverse events such as cytokine release syndrome, making CAR T cell therapy safer and more effective. The benefits of combining rapamycin and TA have been demonstrated in clinical studies shown in FIGS. 4-12. While more research is needed to fully understand the mechanisms behind this approach and to optimize its use in patients, the results are promising and suggest that this combination can be a valuable tool in the fight against cancer.

The present disclosure relates to methods and compositions for optimizing the use of rapamycin in T cell therapy by achieving a balance between its potential advantages in improving T cell survival and reducing adverse effects, while minimizing any negative impact on T cell activation and function. One example of this approach involves combining the CoupledCAR technique with rapamycin treatment to enhance the long-term efficacy of T cell therapy.

The compositions described herein include one or more carriers or pharmaceutically acceptable carriers.

The term “carrier” refers to a diluent, adjuvant {e.g., Freund's adjuvant (complete and incomplete)), excipient, or vehicle with which the therapeutic is administered. Pharmaceutical carriers can be sterile liquids, such as water and oils, including those of petroleum, animal, vegetable or synthetic origin, such as peanut oil, soybean oil, mineral oil, sesame oil and the like Water is a preferred carrier when the pharmaceutical composition is administered intravenously. Saline solutions and aqueous dextrose and glycerol solutions can also be employed as liquid carriers, particularly for injectable solutions.

The term “pharmaceutically acceptable” means approved by a regulatory agency of the U.S Federal or a state government or the EMA (European Medicines Agency) or listed in the U.S. Pharmacopeia Pharmacopeia (United States Pharmacopeia-33/National Formulary-28 Reissue, published by the United States Pharmacopela Convention, Inc., Rockville Md., publication date. April 2010) or other generally recognized pharmacopera for use in animals, and more particularly in humans.

Embodiments relate to a method of enhancing anti-tumor activities of modified cells, the method comprising: administering an effective amount of the modified cells to a subject having a solid tumor; and administering an effective amount of an agent to the subject, the agent comprising rapamycin, wherein the modified cells inhibit growth of the solid tumor in the subject, and wherein the anti-tumor activities in the subject are greater than those in a subject that is administered with an effective amount of modified cells but without the agent.

In embodiments, the modified cells comprise T cells or NK cells, or a combination thereof.

In embodiments, the modified cells comprise a chimeric antigen receptor (CAR) binding a solid tumor antigen.

In embodiments, the solid tumor antigen comprises tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, MAGE A4, EGFR, or a combination thereof.

In embodiments, the solid tumor antigen comprises tMUC1, ACPP, TSHR, GUCY2C, UPK2, CLDN18.2, PSMA, DPEP3, CXCR5, B7-H3, MUC16, SIGLEC-15, CLDN6, Muc17, PRLR, MAGE A4, FZD10, or a combination thereof.

In embodiments, the solid tumor antigen comprises tMUC1, ACPP, TSHR, GUCY2C, UPK2, MAGE A4, CLDN18.2, or a combination thereof.

In embodiments, the CAR comprises an antigen binding domain, a transmembrane domain, a co-stimulatory domain, and a CD3 zeta domain.

In embodiments, the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof; and/or wherein the first CAR comprises a scFv binding CD19, an intracellular domain of 4-1BB or CD28, and CD3 zeta domain, and the second CAR comprises a scFv binding tMUC1, ACPP, TSHR, GUCY2C, or CLDN18.2, an intracellular domain of 4-1BB, CD28, or CD3 zeta domain.

In embodiments, the modified cells comprise a first population of cells comprising a first CAR binding a first antigen, and a second population of cells comprising a second CAR binding a second antigen, and wherein the second antigen is different from the first antigen.

In embodiments, the first antigen comprises a cell surface molecule of a white blood cell (WBC).

In embodiments, the WBC comprises a granulocyte, a monocyte, a lymphocyte, or a combination thereof.

In embodiments, the WBC is a B cell.

In embodiments, the cell surface molecule of the WBC comprises CD19, CD22, CD20, BCMA, CD5, CD7, CD2, CD16, CD56, CD30, CD14, CD68, CD11b, CD18, CD169, CD1c, CD33, CD38, CD138, CD13, or a combination thereof.

In embodiments, the cell surface molecule of the WBC comprises CD19, CD20, CD22, BCMA, or a combination thereof.

In embodiments, the cell surface molecule of the WBC comprises CD19 or BCMA.

In embodiments, the modified cells comprise a vector encoding at least one or more of IL6, IL12, IL-15, IL-7, TNF-α, or IFN-γ.

In embodiments, the modified cells comprise T cells comprising a modified TCR.

In embodiments, the administering an effective amount of the agent to the subject comprises administering an effective amount of rapamycin to the subject at a dose of about 0.6-60 mg per subject.

In embodiments, the administering an effective amount of the agent to the subject comprises administering an effective amount of rapamycin to the subject at a dose of about 1-16 mg per subject.

In embodiments, the administering an effective amount of the agent to the subject comprises administering an effective amount of rapamycin to the subject at a dose of about 1-10 mg per subject.

In embodiments, the administering an effective amount of the agent to the subject comprises administering an effective amount of rapamycin to the subject at a dose of about 1-6 mg per subject.

In embodiments, the administering an effective amount of the agent to the subject comprises administering an effective amount of rapamycin to the subject more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the subject has been administered the effective amount of the modified cells.

In embodiments, the administering an effective amount of the agent to the subject comprises administering an effective amount of rapamycin to the subject more than about 10-20 days after the subject has been administered the effective amount of the modified cells.

In embodiments, the administering an effective amount of the agent to the subject comprises administering an effective amount of rapamycin to the subject about the CAR T cell number reach to a peak after the subject has been administered the effective amount of the modified cells.

In embodiments, the method further comprises monitoring a concentration of CAR T cells in the blood of the subject; determining one or more days corresponding to a peak or peaks of the concentration of CAR T cells in the blood; and/or administering the effective amount of the agent abound the one or more days.

In embodiments, the method further comprises monitoring a concentration of CAR T cells in the blood of the subject. There are several methods for monitoring circulating CAR T cells in patients' blood, including flow cytometry, qPCR, and ELISA. Flow cytometry is a commonly used technique for monitoring CAR T cells in patients' blood. It involves labeling the CAR T cells with fluorescent markers that can be detected by flow cytometry. The labeled cells can be quantified and their phenotypic characteristics can be analyzed. qPCR method involves detecting the DNA of CAR T cells in patients' blood using quantitative polymerase chain reaction (qPCR). This can provide a quantitative measure of the number of CAR T cells in the blood. Enzyme-linked immunosorbent assay (ELISA) is another method for monitoring CAR T cells in patients' blood. It involves detecting specific proteins produced by the CAR T cells, which can be quantified to determine the number of CAR T cells present in the blood.

In embodiments, the method further comprises determining the peak of CAR-T in the patients' blood based on the monitoring data. The data collected from the monitoring can be used to plot a time course of the number of CAR T cells in the blood. This can be used to identify the time point at which the CAR T cells peak (e.g., between 7-21 days after infusion). In embodiments, the effective amount of the agent may be administered before one or more days (e.g., 1-2 days) before the peak or after the peak.

In embodiments, administering the agent to manage the number of circulating CAR T cells in a patient may be necessary in certain cases to avoid adverse events such as cytokine release syndrome (CRS). The timing and dosing of such drugs will depend on the specific situation and the individual patient's response to therapy. For example, based on the monitoring data, the peak of CAR T cells in the patient's blood can be used to determine when to administer drugs to manage their number. In embodiments, the agent may be administered after the peak of CAR T cells in the blood, as this is the time when the risk of CRS is highest. In embodiments, rapamycin can be administered once daily for several days, starting at the time of the CAR T cell peak in the blood. The dosage and duration of rapamycin treatment will depend on the patient's response to therapy and the severity of CRS symptoms. In embodiments, administering the agent to manage the number of circulating CAR T cells in a patient may be associated with a level of CRS.

In embodiments, tracking the decline in the number of CAR-T cells in the patient's blood can provide a more accurate estimate of how long the cells will persist in the patient's system. The agent may be administered about the time of the decline. In embodiments, the peak of circulating CAR-T cells may be determined without tracking their decline. Based on the clinical trial data you provided, the peak of CAR-T cells was observed between 7-21 days after infusion. This suggests that in most patients, the peak of circulating CAR-T cells is likely to occur within this timeframe. In addition to the timing of infusion, other factors such as the dose of CAR-T cells, the rate of CAR-T cell proliferation, and the rate of CAR-T cell clearance from the blood may also influence the timing of the peak. By taking these factors into account, a more accurate prediction of the peak of circulating CAR-T cells in a given patient may be made, even without tracking their decline. In embodiments, the timing of the peak may vary between patients, and other factors such as the dose of CAR-T cells, the rate of CAR-T cell proliferation, and the rate of clearance from the blood may also influence the timing of the peak. Furthermore, it is also possible that the peak may occur outside of this timeframe in some patients. This may be due to individual differences in the patient's immune system, the characteristics of the cancer being targeted, or other factors that can impact the response to CAR-T cell therapy.

In embodiments, the method further comprises measuring the anti-tumor activities 20, 30, 40, 50, 60, 70, 80, 90, or 120 days after the subject has been administered the effective amount of the modified cells, wherein the anti-tumor activities in the subject are greater than those in a subject that is administered with an effective amount of modified cells but without the agent 20, 30, 40, 50, 60, 70, 80, 90, or 120 days after the subject has been administered the effective amount of the modified cells.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

Embodiments related to a method of modulating CAR T-cell therapy in a patient diagnosed with cancer, the method comprising administering a therapeutically effective amount of CAR T-cells and a regimen of rapamycin wherein the rapamycin is administered to achieve a blood concentration that reduces activation markers in the CAR T-cells without inhibiting their proliferation. In embodiments, the activation markers include CD137 and CD40L, and their reduction is quantified by flow cytometry analysis.

In embodiments, Rapamycin is dosed in a manner that specifically preserves or enhances the expression of CD62L on CAR T-cells, indicating the promotion of a memory phenotype that is crucial for sustained antitumor activity.

In embodiments, the administration of Rapamycin is carefully calibrated to achieve a plasma concentration within the specific range of 5 ng/ml to 15 ng/ml 24 hours after administration, optimizing the immunomodulatory impact on CAR T-cells.

In embodiments, the administration of Rapamycin is strategically timed either before, concurrently with, or after the CAR T-cell therapy. This timing is optimized based on the activation state of the CAR T-cells to maximize therapeutic efficacy.

In embodiments, the effectiveness of the Rapamycin regimen is determined by evaluating the decrease in cytokine release from CAR T-cells. This assessment ensures that Rapamycin's modulation does not adversely affect the proliferative capacity of the CAR T-cells.

In embodiments, the patient's tumor response to CAR T-cell therapy is continuously monitored, allowing for the Rapamycin dosage to be adjusted as necessary. This ensures that the antitumor activity is maximized based on real-time response data.

In embodiments, the dosing of Rapamycin is tailored based on a patient-specific response profile, including changes in PD1 and CD62L expression on CAR T-cells. This personalized approach optimizes the balance between efficacy and safety.

In embodiments, alongside Rapamycin, other immunomodulatory agents are administered that complement the action of Rapamycin without interfering with the CAR T-cell modulation, enhancing the overall therapeutic strategy.

In embodiments, the CAR T-cells are genetically engineered to target specific antigens associated with the patient's cancer type. Rapamycin is used in conjunction with these targeted CAR T-cells to modulate their activity for improved treatment outcomes.

Embodiments relate to a method of modulating anti-tumor activities in patients with solid tumors through administering modified cells, including T cells, NK cells, or a combination thereof, along with a regimen of rapamycin. The modified cells are designed to inhibit tumor growth more effectively when combined with rapamycin than when administered alone.

Modified cells may include an exogenous nucleic acid encoding IFNγ, enhancing IFNγ sensitivity in the tumor and improving anti-tumor efficacy. Additionally, these cells may express a chimeric antigen receptor (CAR) targeting solid tumor antigens such as tMUC1, PRLR, CLCA1, MUC12, GUCY2C, GPR35, and others, enabling precise targeting of tumor cells.

The CAR structure in these cells includes an antigen-binding domain, a transmembrane domain, a co-stimulatory domain, and a CD3 zeta domain, with the co-stimulatory domain potentially comprising CD27, CD28, 4-1BB, OX40, among others, to enhance the cells' activation and survival. Moreover, the therapeutic strategy may involve administering a combination of cells equipped with different CARs targeting diverse tumor antigens, addressing tumor heterogeneity.

Rapamycin is dosed to achieve therapeutic blood concentrations that optimize the anti-tumor response, with dosages ranging from 0.6-60 mg per subject, tailored to the patient's needs. The timing of rapamycin administration is carefully selected to coincide with peak CAR T-cell proliferation, enhancing therapeutic efficacy.

The method includes monitoring CAR T-cell concentrations in the patient's bloodstream, determining optimal rapamycin administration timing based on CAR T-cell population peaks, and evaluating enhanced anti-tumor activities at various post-administration intervals.

This approach offers a detailed framework for using modified cells and rapamycin in a complementary fashion to significantly improve outcomes in patients with solid tumors, providing a nuanced strategy for enhancing the effectiveness of cancer immunotherapy.

Related sequences, compositions, and methods of treating cancer are provided in this Application and Innovative Cellular Therapeutics' PCT Patent Publication NOS: WO2016138846, WO2018126369, WO2017167217, WO2019140100, WO2020146743, WO2021216731, WO2020106843, WO2020047306, and WO2022150831 and US Patent Publication NOS: US20210060069 and US20210100841, which are incorporated by reference in their entirety.

The following examples illustrate exemplary methods provided herein. These examples are not intended, nor are they to be construed, as limiting the scope of the disclosure. It will be clear that the methods can be practiced otherwise than as particularly described herein. Numerous modifications and variations are possible in view of the teachings herein and, therefore, are within the scope of the disclosure.

### EXAMPLES

Lentiviral vectors that encode individual CAR molecules were generated and transfected with T cells, which are discussed below. In addition, techniques related to cell cultures and the construction of cytotoxic T lymphocyte assay may be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in its entirety.

It was observed by Applicant that activation of CD19 CAR T cells promoted the expansion of non-transduced T cells, indicating that expansion may apply to CAR T cells targeting solid tumors. The non-transduced T cells were replaced with PAP CAR T cells targeting prostate cancer. In vitro experiments show that CD19 CAR T cells can, by killing B cells, promote PAP CAR T cells to expand and release cytokines.

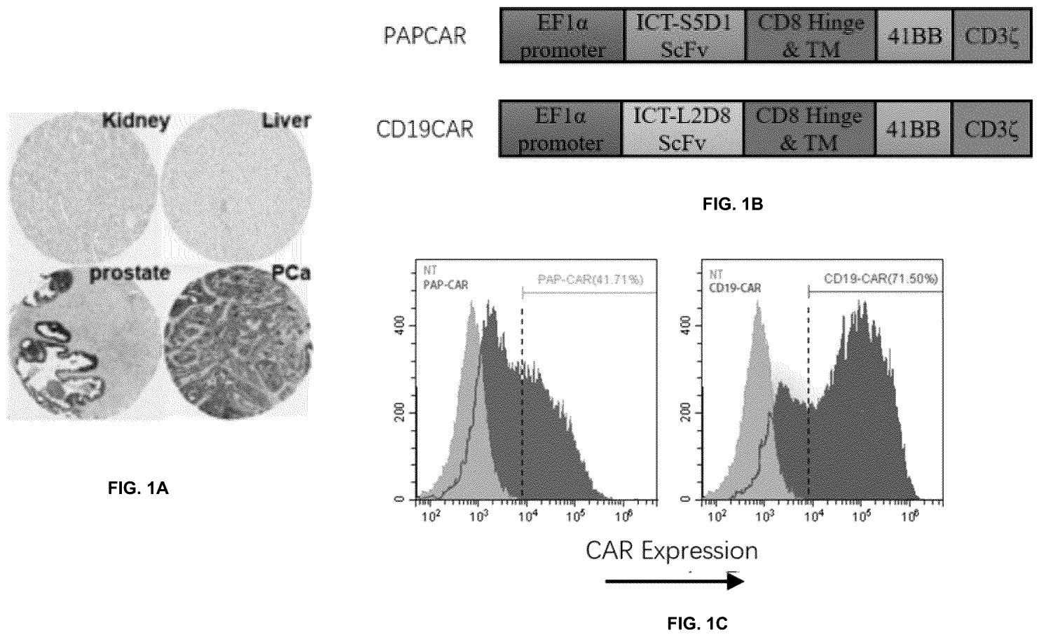

FIGS. 1A-1C show constructs and expression of CD19-CAR and PAP-CAR in corresponding T cells. FIG. 1A shows PAP expression in kidney, liver, prostate, and prostate cancer samples. Immunohistochemical staining shows that PAP is highly expressed in prostate cancer tissue and is only expressed in prostate glands in normal tissues. FIG. 1B shows constructs of vector encoding PAP CAR and vector encoding CD19 CAR. FIG. 1C shows CAR expression of CD19 CAR and PAP CAR. CAR expression shows that the proportion of CD19 CAR expression is 71.50%, and the proportion of PAP CAR expression is 41.71%.

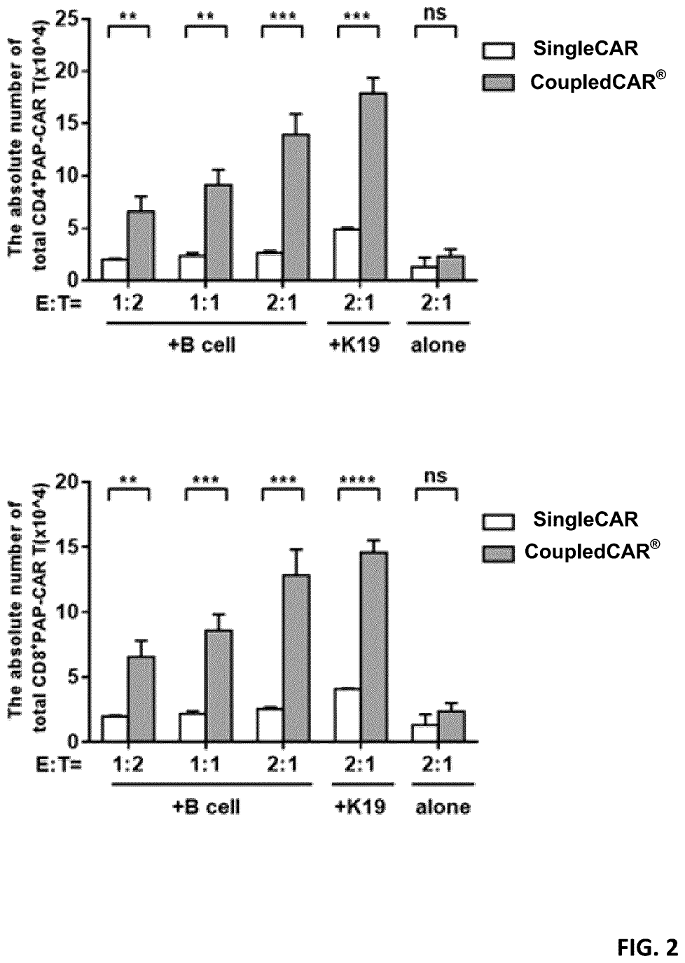

FIG. 2 shows expansion of PAP CAR T cells in various culturing systems. Corresponding cells were co-cultured for 96 hours using Cell Trace and gate PAP CAR+CD4+/CD8+. SingleCAR refers to PAP CAR T+NT group; CoupledCAR® refers PAP CAR+CD19 CAR group; and “alone” refers to cells without substrate added in the co-culture system as negative controls. The experimental group included B cells with different CD19 CAR ratios. PAP CAR T cells were labeled with CFSE and placed in different systems for culturing. After 96 hours, the expansion of PAP CAR T cells was determined. The vertical axis is the absolute number of CD4/CD8 positive PAP CAR T cells after expansion. Ratio of CD19 CAR+: B cell is 2:1/1:1/2:1. The expansion of the group of CoupledCAR with B cells was significantly higher than the group without B cells. Statistics on total CD4/CD8 T cells show CD19 CAR T cells can significantly promote the expansion of PAP CAR T cells, and the effect of the expansion is stronger as the proportion of CD19 CAR increases. When E:T (Effector to Target ratio) is 2:1, the expansion of PAP CAR in the CoupledCAR and B group was approximately 5 times that of the control group, and the results were consistent in CD4/CD8 T cells.

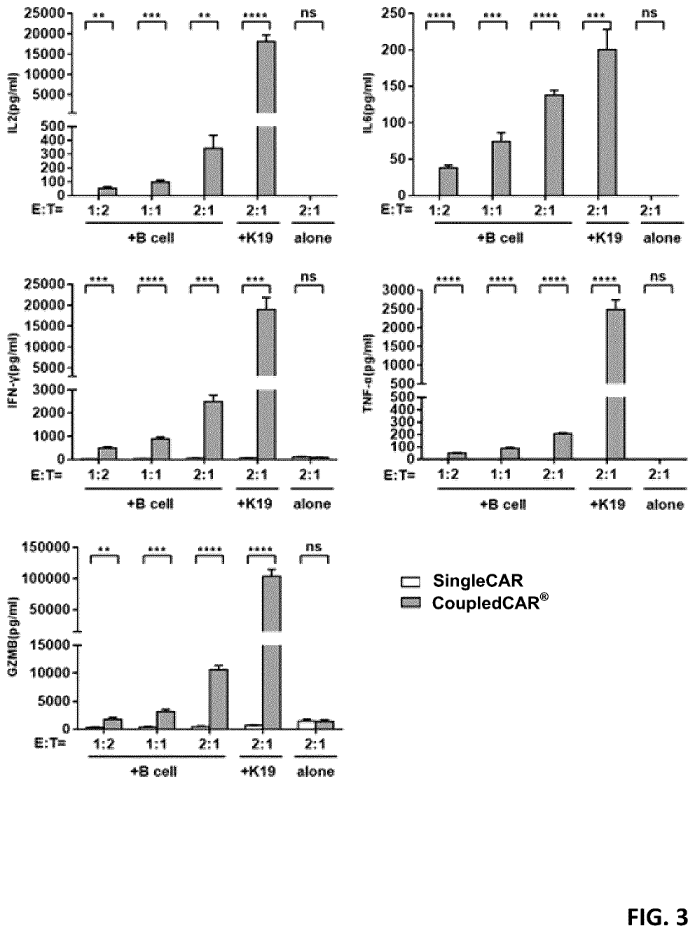

FIG. 3 shows cytokine release analysis of co-cultured cells with respect to PAP CAR and CD19 CAR. Corresponding cells were co-cultured for 48 hours, and cell supernatant was collected to determine cytokines. CD19 CAR T cells mediate the release of various cytokines in the presence of B cells, and the higher the proportion of CD19 CAR, the more of the cytokines are released, indicating that CD19 CAR promotes the expansion of PAP CAR T cells.

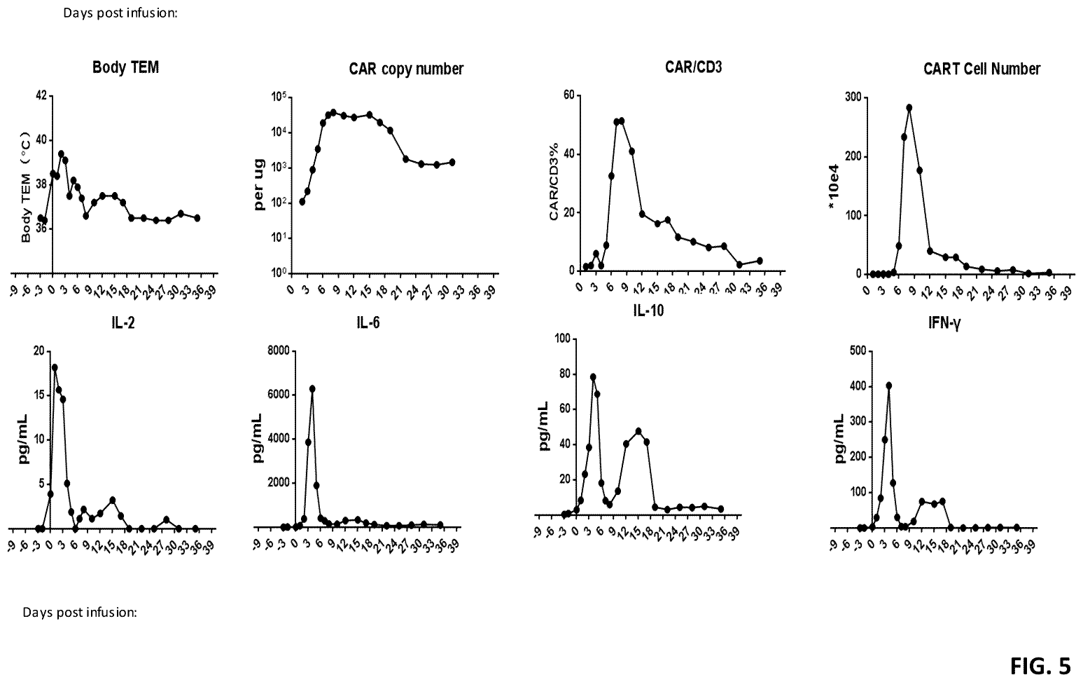

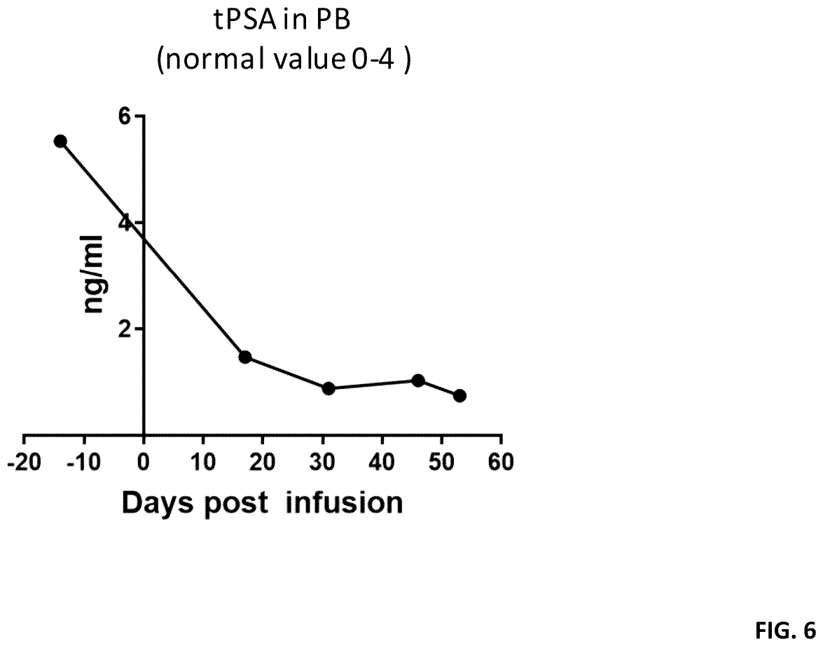

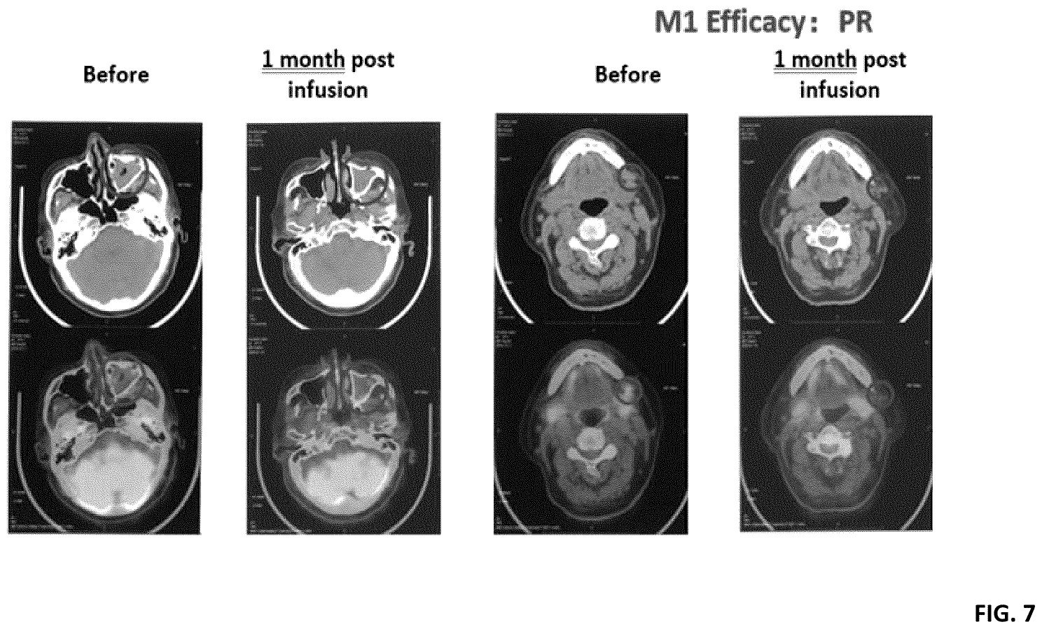

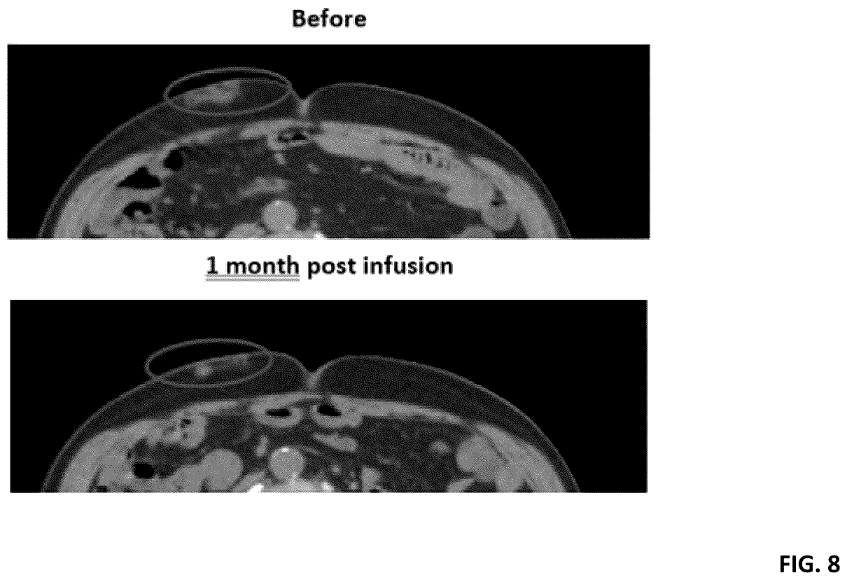

FIG. 4 shows the protocol for the treatment using PAP CAR T cells. FIG. 5 shows cytokine release in response to the infusion of cells including PAP CAR T cells. FIG. 6 shows tPSA assay in the peripheral blood of the patient. FIGS. 7 and 8 show PET-CT scanning images of the patient one month after the cell infusion.

TABLE 3
Clinical trial data

| Patient's | | | | |
|---|---|---|---|---|
| ID | Cancer | Infusion CART/kg | CSR > 2 | Efficacy |
| 05 | r/r prostate | 2.12 × 10⁶ | No | PR |
| | adenocarcinoma | | | |

TABLE 4
Preparation of cells for clinical trials

| Patient's | | Infusion | |
|---|---|---|---|
| ID | Vectors | Methods | Pre-treatment |
| 05 | Vector 1: PAP-CAR; Vector 3: hCD19-CAR- | Fresh cells | FC regimen at −2 |
| | NATF-IL12-VHL; and Vector 4: hCD19- | | days |
| | CAR-NATF-IFNγ; and Vector 5: hCD19- | | (cyclophosphamide |
| | CAR-NATF-IL6 | | 500 mg/m2, |
| | | | fludarabine 30 |
| | | | mg/m2) |

Rapamycin is utilized in T cell therapy to manage T cell activation and growth. It operates by binding to mTOR, a protein complex that plays a crucial role in regulating cellular metabolism, growth, and proliferation. By blocking mTOR, Rapamycin slows down T cell activation and growth, improving the safety of T cell therapy and reducing the likelihood of side effects. However, some studies have shown that rapamycin may also decrease the effectiveness of T cell therapy by hindering T cell activation, which is essential for their proper functioning.

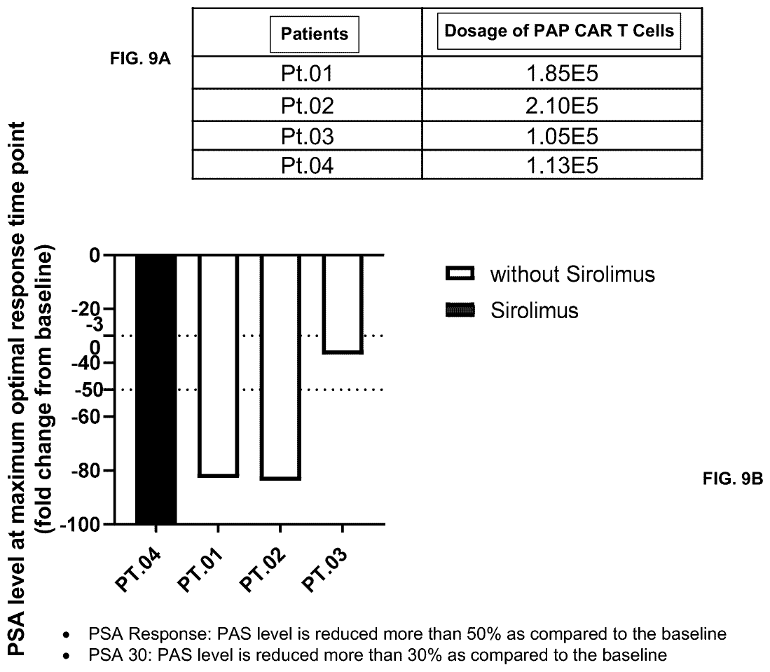

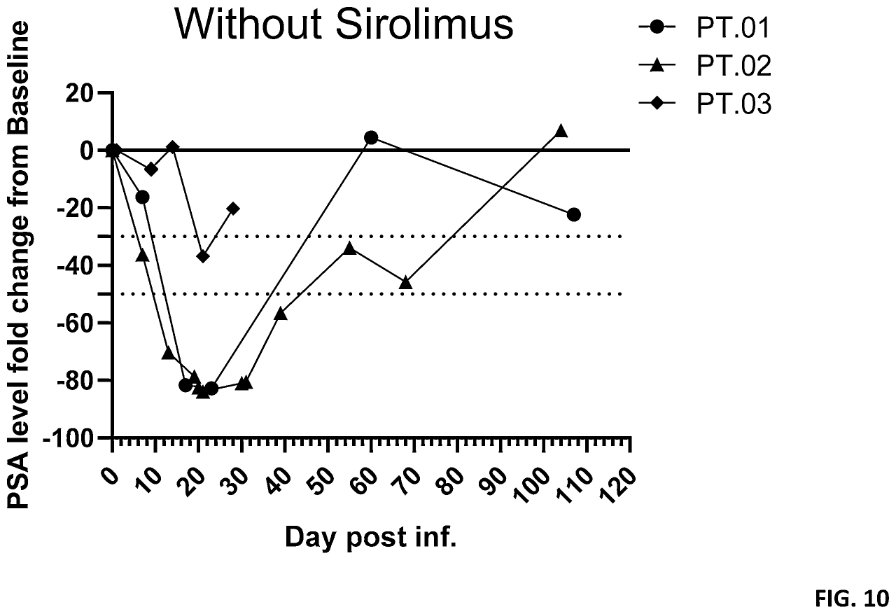

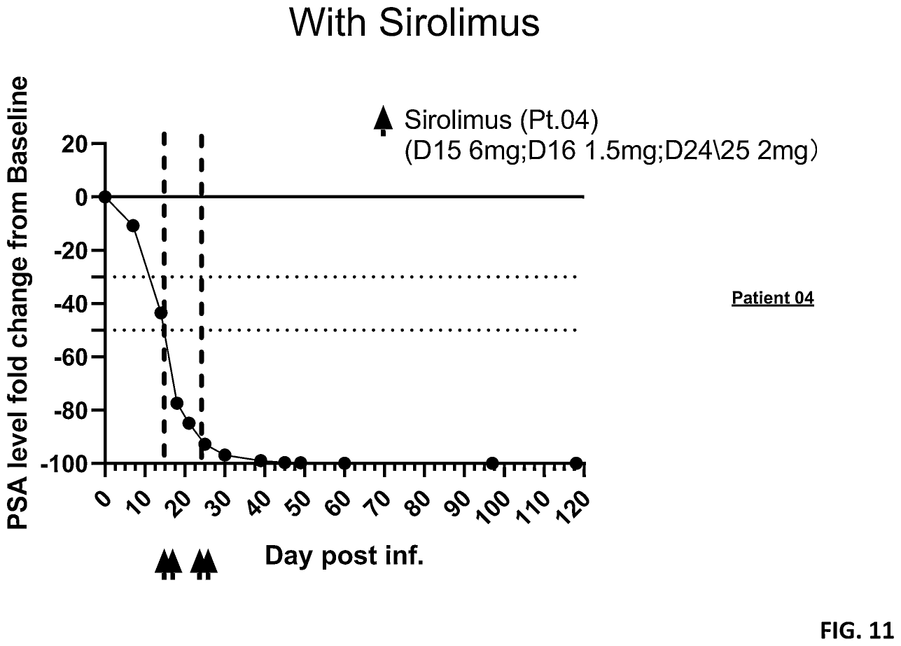

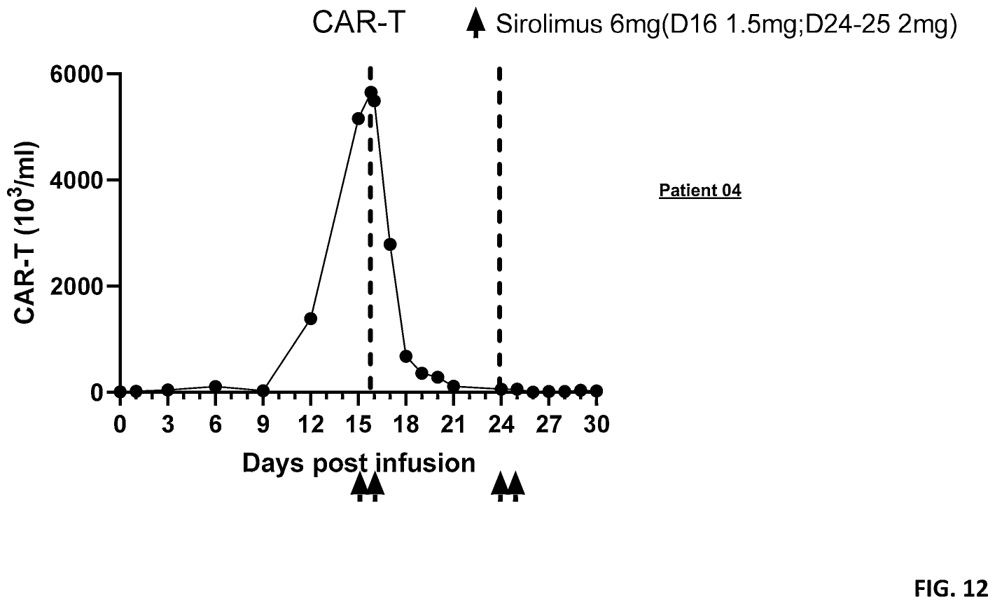

To investigate the impact of rapamycin on CAR T cell therapy, patients were separated into two groups. Group 2, consisting of Patient 04, received rapamycin treatment, while Group 1, made up of Patients 01-03, did not. These patients were infused with PAP CAR T cells using the protocol described above. FIG. 9A shows dosages of CAR T cells infused into the patients. FIG. 9B shows PSA level changes before and after CAR T cell infusion. As shown in FIG. 9B, Patient 04 who received rapamycin showed the best PSA response. FIG. 10 depicts the fluctuation of PSA levels over time for Group 1. FIG. 11 illustrates the variation of PSA levels over time for Group 2. As shown in FIGS. 10 and 11, the PSA level of Patient 04, who received rapamycin treatment, was stabilized at a normal level for an extended period, while Patients 01-03 did not experience a sustained normal PSA level. FIG. 12 shows the scheme of administration of rapamycin to Patient 04 after administration of CAR T cells.

A patient was infused with mixed T cells comprising GCC CAR T and CD19 CAR T cells. More information about the infusion and CAR T cells can be found in PCT Patent Application No: WO2020146743 as well as US Patent Publication No: US20210100841, which are incorporated by their entirety. Biopsy was obtained before and after the cell infusion, and single cell sequence and analysis were performed.

Single-cell RNA-seq (ScRNA-seq) experiment was performed in the laboratory of NovelBio Bio-Pharm Technology Co., Ltd. The tissues were surgically removed and kept in MACS Tissue Storage Solution (Miltenyi Biotec) until processing. The tissue samples were processed as described below. Briefly, samples were first washed with phosphate-buffered saline (PBS), minced into small pieces (approximately 1 mm3) on ice and enzymatically digested with 200 U/mL collagenase I (Worthington), 100 U/mL collagenase IV (Worthington) and 30 U/mL DNase I (Worthington) for 20 min at 37° C., with agitation. After digestion, samples were sieved through a 70 μm cell strainer, and centrifuged at 300 g for 5 minutes(mins). After the supernatant was removed, the pelleted cells were suspended in red blood cell lysis buffer (Miltenyi Biotec) to lyse the red blood cells. Whole blood was also prepared by treatment of the peripheral blood with red blood cell lysis buffer (Miltenyi Biotec). After washing with PBS containing 0.04% BSA, the cell pellets were re-suspended in PBS containing 0.04% BSA and re-filtered through a 35 μm cell strainer. The single-cell suspension was then stained with AO/PI for viability assessment using Countstar Fluorescence Cell Analyzer.

The scRNA-Seq libraries and V(D)J libraries were generated using the 10× Genomics Chromium Controller Instrument and Chromium Single Cell 5′ library & gel bead kit, along with the V(D)J enrichment kit (10× Genomics, Pleasanton, CA). Briefly, cells were concentrated to approximately 1000 cells/uL and loaded into each channel to generate single-cell Gel Bead-In-Emulsions (GEMs). After the reverse transcription (RT) step, GEMs were broken and barcoded-cDNA was purified and amplified. The amplified barcoded cDNA was used to construct 5′ gene expression libraries and TCR enriched libraries. For 5′ library construction, the amplified barcoded cDNA was fragmented, A-tailed, ligated with adaptors, and index PCR amplified. For the V(D)J library, human T cell V(D)J sequences were enriched from the amplified cDNA followed by fragmentation, A-tailing, adaptor ligation and index PCR amplification. The final libraries were quantified using the Qubit High Sensitivity DNA assay (Thermo Fisher Scientific) and the size distribution of the libraries was determined using a High Sensitivity DNA chip on a Bioanalyzer 2200 (Agilent). All libraries were sequenced by illumina sequencer (Illumina, San Diego, CA) on a 150 bp paired-end run.

The second-generation high-throughput sequencing data were aligned and quantified using the Cell Ranger Single-Cell Software Suite (version 3.0.2, 10× Genomics) against the GRCh38 human reference genome. Unique molecular identifier (UMI) counts were summarized for each cell of each gene and converted into a Seurat object by the R package Seurat (version 4.0). Quality of cells were then assessed based on three metrics step by step: (1) The number of total UMI counts per cell (200-4000); (2) The number of detected genes per cell (1,600-25,000); (3) The proportion of mitochondrial gene counts (<25%). After exclusion of low-quality cells, 21,396 protein-coding genes across 12,245 single cells remained for downstream processing.

The TCR-seq data was processed using Cell Ranger (version 3.0.2) against the human VDJ reference genome. In all TCR contigs assembled, if two or more cells had identical apha-beta pairs, the apha-beta pair were identified as clonal TCRs, and these T cells were identified as clonal T cells. To integrate TCR results with the gene expression data, the TCR-based analysis was performed only for cells that were identified as T cells.

After quality control, raw UMI counts were lognormalized using the scale of 10,000. To cluster single cells by their expression, an unsupervised graph-based clustering algorithm implemented in Seurat v4 (version 4.0) was used. Single cells of four samples from this patient were integrated and embedded into a shared low-dimension space through integrated analysis (CCA) by the Seurat v3 function IntegrateData. The highly variable genes were generated with appropriate threshold of the mean expression and dispersion (variance/mean). Principal component analysis (PCA) was performed on about 2000 variable genes. The function FindClusters on 30 PCs with resolution 0.6 was used to perform the first-round cluster. Each cell cluster was annotated by the exceptionally high amounts expression of canonical marker genes. For visualization, the dataset dimensionality was reduced using the Barnes-Hut t-Distributed Stochastic Neighbor Embedding (t-SNE).

All statistical analyses were conducted using R software (R Foundation for Statistical Computing). Gene set variation analysis implemented in the GSVA package (version 1.3.0) was used for gene set enrichment analysis. Comparisons between two groups of samples were evaluated using Wilcoxon ranksum test (Mann-Whitney U-test) for statistical analysis. * P<0.05, ** P<0.01, *** P<0.001.

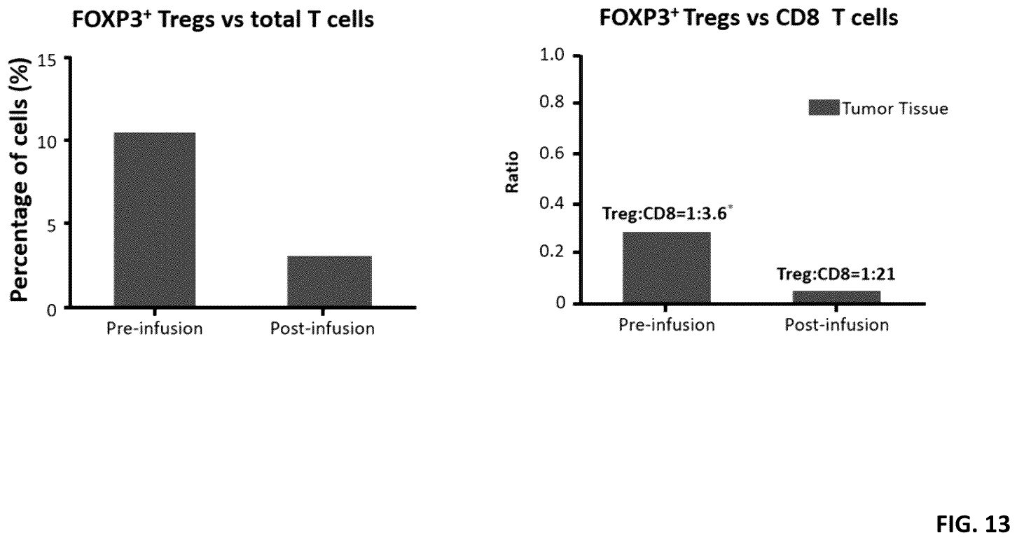

FIG. 13 shows T regulator cells (Tregs) were reduced in tumor tissue. 1. Treatment with CAR T cells also had a dramatic impact on tumor-infiltrating CD4+ T cell subsets. The population of FOXP3+ T cells (Treg) were especially reduced compared to pre-infusion groups. 2. The ratio of Tregs to CD8+ T cells in pre-infusion was at 1:3.6, which demonstrated that Tregs may manifest suppression of effector T cell proliferation via a reduction in division destiny in the effector T cell population, as described in the reference. Strikingly, the reduction of Tregs after CAR T cell infusion has led to higher Tregs to CD8+ T cells ratio at 1:21, indicating less inhibition on CD8+ T cells in solid tumor tissue.

The experiments were conducted to evaluate rapamycin's effect on certain markers' expression in CAR T cells. CAR T cells were cultured and treated under various conditions. The control group (1234 (CD19 CAR T cells) alone) had no additional treatment. The positive control group (1234+nalm6) included stimulation with nalm6 cells. Three experimental groups were treated with increasing concentrations of rapamycin: ⅓ Cmax, Cmax, and 3×Cmax, in combination with nalm6 stimulation. When taken orally at a dose of 2 mg/day, the maximum blood concentration (Cmax) is reached.

Post-treatment, cells were assessed for the expression of activation and memory markers. Results are shown here: CAR+/CD137+: CAR T cells showed a reduction in the activation marker CD137 as rapamycin concentration increased; CAR+/CD40L+: A similar trend was observed for the activation marker CD40L, with a decrease in expression correlating with higher rapamycin levels; CAR+/PD1+: The expression of the exhaustion marker PD1 was also reduced following rapamycin treatment, suggesting a potential reduction in T-cell exhaustion; CD62L+/CD45RO+: Memory T-cell markers CD62L and CD45RO were differentially expressed, with an increase in CD62L, indicating a potential shift towards a memory phenotype with rapamycin treatment. Overall, the data suggest that rapamycin modulates CAR T cells' activation and memory status, potentially improving their function by reducing exhaustion markers and enhancing memory cell populations. The experimental concentrations used would be approximate: ⅓ Cmax: ⅓ of 16, which would be about 5.33 nM; Cmax: the mentioned value of 16 nM; 3×Cmax: three times 16 nM, which would equal approximately 48 nM.

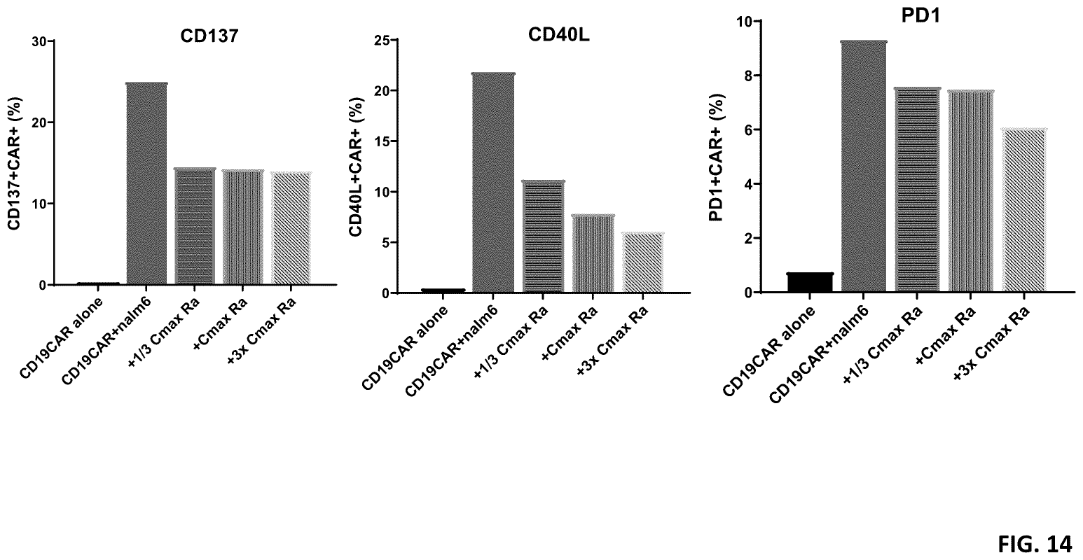

FIG. 14 shows the impact of rapamycin on CAR T cell activation and Exhaustion Markers. The bar graphs display the percentage of CAR T cells expressing key markers under different treatment conditions. Each graph corresponds to a different marker: CD137: Activation marker CD137 expression across five treatment conditions: CD19CAR alone, CD19CAR+nalm6, and rapamycin at ⅓ Cmax, Cmax, and 3× Cmax; CD40L: Activation marker CD40L expression under the same five conditions, indicating the inhibitory effect of rapamycin; PD1: Exhaustion marker PD1 expression, demonstrating a decrease in PD1 with increasing rapamycin concentration. These graphs collectively illustrate the dose-dependent effects of rapamycin on CAR T cell activation and potential exhaustion.

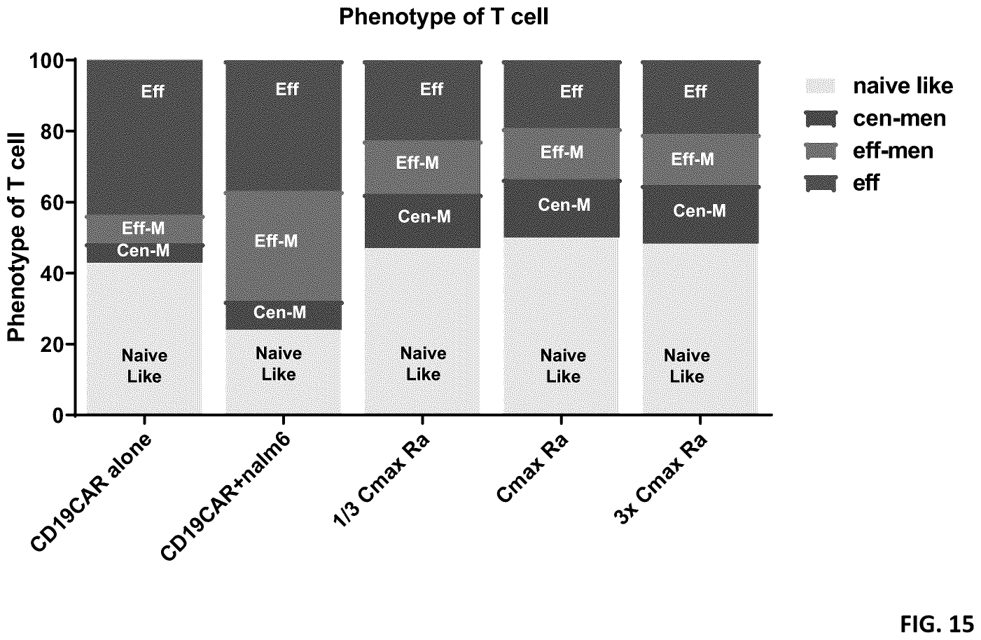

FIG. 15 shows the distribution of T-cell phenotypes under varied treatment conditions. The stacked bar graph represents the distribution of T cell phenotypes following different treatment regimens. Each color within the bars corresponds to a subtype of T cell: Naive-like (yellow): T cells with a naive phenotype; Central memory (Cen-M, red): T cells with characteristics of central memory; Effector memory (Eff-M, green): T cells with properties of effector memory; Effector (Eff, purple): Fully differentiated effector T cells. The treatment conditions from left to right are CD19CAR-alone: CAR T cells without additional treatment; CD19CAR+nalm6: CAR T cells with nalm6 stimulation; ⅓ Cmax Ra: CAR T cells treated with ⅓ the peak concentration of rapamycin; Cmax Ra: CAR T cells treated with the peak concentration of rapamycin; 3×Cmax Ra: CAR T cells treated with three times the peak concentration of rapamycin. This graph visually conveys how each treatment influences the composition of T cell subtypes, indicating the potential impact on the immune response and memory formation.

The following study investigated the effects of rapamycin on CAR T cells and tumor cell lines. In the first part of the experiment, cytokine release assays were performed. The results showed that the addition of rapamycin led to a significant decrease in cytokine release (IL-2, TNF-α, IFN-γ, GZMB) from CAR T cells, with a dose-dependent relationship: higher rapamycin concentrations led to lower cytokine release. In the same experiments, T cell proliferation was assessed to determine whether rapamycin affects the expansion of CAR T cells. Using CD19 CAR alone as a negative control and nalm6 cell stimulation for expansion, it was found that after 96 hours, despite some degree of activation suppression by rapamycin, the cell proliferation was not inhibited and, to some extent, was increased. This suggests that rapamycin does not negatively impact CAR T cell proliferation in vitro over a longer timeline and might even promote it, possibly due to an increased proportion of memory cells. In the last part of the study, rapamycin was directly applied to two types of tumor cell lines, PC3 and LNCAP, to assess its impact on tumor cell proliferation. Post-treatment, both tumor cell lines showed a degree of proliferation inhibition. This was measured using a cell counting method after digestion with trypsin, indicating that all three rapamycin concentrations reduced tumor cell numbers. In summary, while rapamycin can suppress certain functions of CAR T cells, such as cytokine release, it does not inhibit their proliferation. It can decrease the proliferation of tumor cells, aligning with clinical observations.

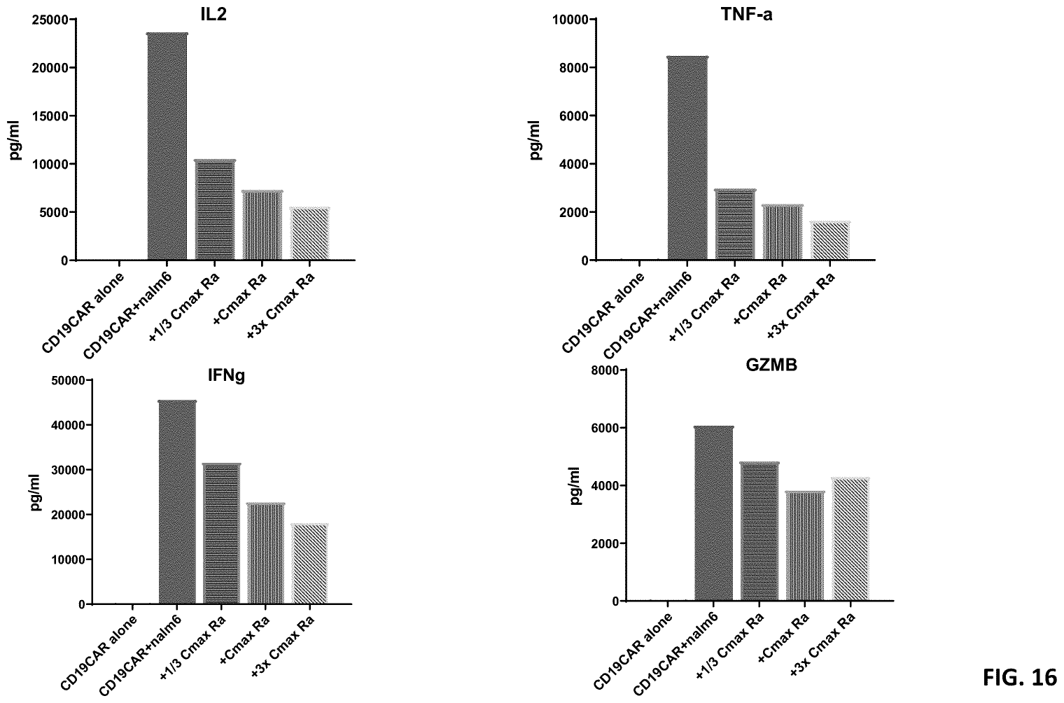

FIG. 16 shows the effects of rapamycin on cytokine production by CAR T cells. The bar graphs illustrate the concentration of various cytokines released by CAR T cells under different treatment conditions. Each graph represents a different cytokine: IL-2 shows the concentration of interleukin 2, with a marked decrease in the presence of rapamycin; TNF-α depicts the levels of tumor necrosis factor-alpha, which similarly declines with rapamycin treatment; IFN-γ represents interferon-gamma levels, also reduced by rapamycin; GZMB: Indicates the amount of granzyme B, with a decrease following rapamycin exposure.

For each cytokine, the following conditions are compared: CD19 CAR-alone: Baseline cytokine levels from CAR T cells without additional stimulation or drug treatment; CD19CAR+nalm6: Cytokine levels after stimulation with nalm6; ⅓ Cmax Ra: Cytokine levels with rapamycin treatment at one-third the peak blood concentration; Cmax Ra: Cytokine levels at the peak blood concentration of rapamycin; 3×Cmax Ra: Cytokine levels at three times the peak blood concentration of rapamycin; These graphs demonstrate the dose-dependent inhibitory effect of rapamycin on cytokine release from CAR T cells.

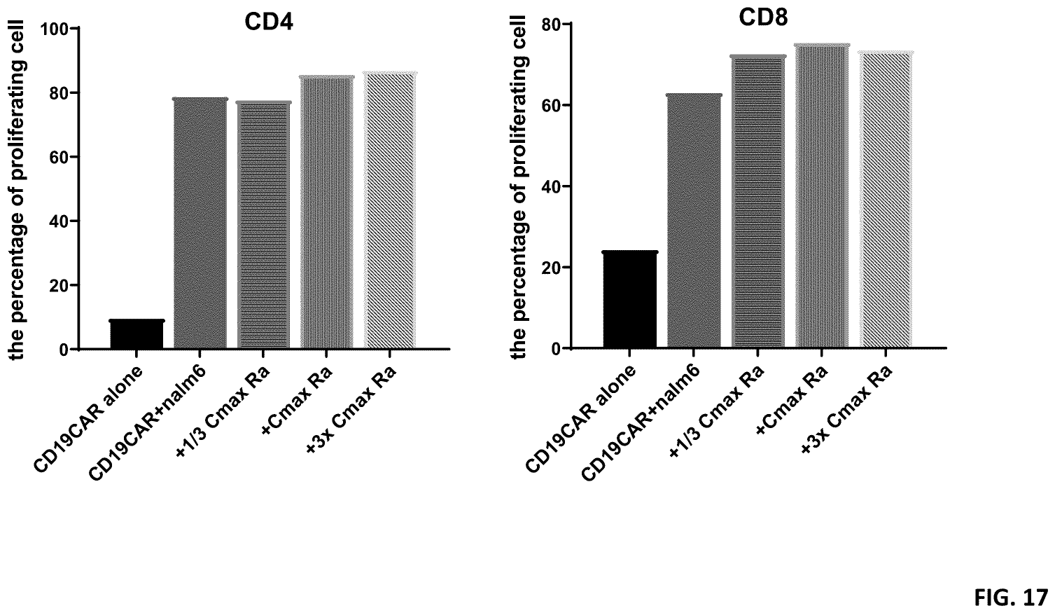

FIG. 17 shows rapamycin's Influence on CAR T cell proliferation. The bar graphs depict the proliferation rates of CAR T cells, specifically CD4+ and CD8+ subsets, after 96 hours of stimulation with CD19 antigen and varying concentrations of rapamycin. CD4: The left graph shows the percentage of proliferating CD4+ CAR T cells across different conditions: CD19 CAR-alone, CD19 CAR+nalm6, and with rapamycin at ⅓ Cmax, Cmax, and 3×Cmax. CD8: The right graph indicates the percentage of proliferating CD8+ CAR T cells under the same treatment conditions as CD4+. In vitro experiments confirmed that rapamycin does not reduce CAR T cell proliferation after 96 hours of stimulation by the CD19 antigen; rather, it may enhance cell proliferation to some extent, potentially due to an increased proportion of memory cells.

The following study details an experiment where prostate cancer tumor cells (PC3) were treated with ⅓ Cmax concentration of rapamycin. The control group was treated with PBS. RT-PCR was used to measure the expression of various genes, including those involved in the IFN pathway, P53, and others. 48 hours of rapamycin treatment shows increased IFNGR1/2, P53, STAT1, STAT2, and B2M gene expression compared to the control (PBS). This suggests that rapamycin upregulates the IFN pathway, including its receptors and downstream signaling via JAK-STAT, and increases the expression of P53 and B2M. 72 hours after removing rapamycin and waiting for an additional 72 hours, the expression levels of most genes returned to levels comparable to the control group, except for the intracellular signaling components (such as JAK-STAT), which remained elevated. This indicates a potential lasting effect of rapamycin on intracellular signaling pathways. The findings suggest that the PC3 tumor cell line becomes more sensitive to IFN-γ signaling following rapamycin treatment, potentially due to the upregulation of IFNGR and components of the antigen-presenting machinery (such as B2M), aligning with the decreased cell numbers observed in the previous data set. The results may also imply an enhanced immunogenicity of the tumor cells, making them more susceptible to immune surveillance. This observation was novel at the time of the experiment, as no literature was found relating rapamycin treatment with increased sensitivity of prostate cancer cells to IFN-γ. In conclusion, rapamycin affects the gene expression profile of prostate cancer cells, with potential implications for their sensitivity to immune-mediated destruction and long-term alterations in signaling pathways.

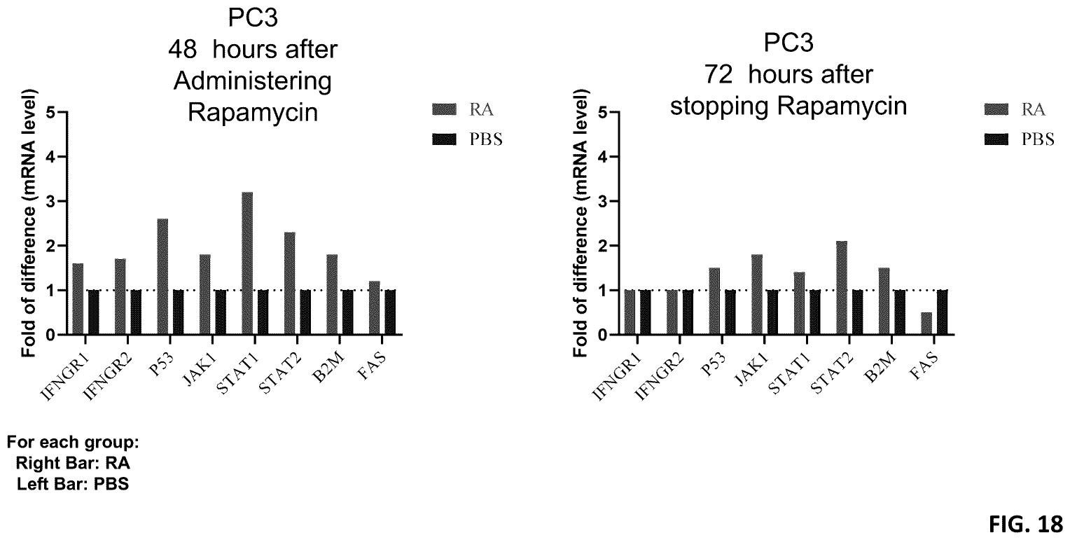

FIG. 18 shows rapamycin upregulates IFN-γ sensitivity in prostate cancer cells. The bar charts compare gene expression in PC3 prostate cancer cells treated with rapamycin (RA) and a control group treated with PBS. Red bars represent RA-treated cells, and blue bars represent control cells. Left Chart (48 hours after administering rapamycin) shows elevated expression of IFNGR1/2, JAK, STAT1, STAT2, B2M, and FAS in rapamycin-treated cells compared to control, indicating upregulation of IFNγ receptors and associated signaling pathways. Right Chart (72 hours after stopping rapamycin) shows post-treatment, IFNGR1/2, and JAK-STAT signaling levels decrease but remain above control levels, suggesting a sustained modulation effect by rapamycin. These observations reveal a novel role for rapamycin in enhancing IFNγ sensitivity in prostate cancer cells, diverging from prior research primarily focused on its mTOR inhibition and cell cycle arrest properties. “Upregulating IFNγ sensitivity in solid tumors” refers to the process of enhancing a tumor's responsiveness to the immune system's IFN-γ signaling, typically through genetic or pharmacological means such as administering rapamycin. This process involves increasing the expression or activity of IFN-γ receptors and related signaling components (e.g., JAK-STAT pathway elements) in tumor cells, making them more susceptible to IFN-γ-mediated immune surveillance, destruction, or both. This strategy aims to boost the effectiveness of immunotherapies, including T cell-based treatments, by making cancer cells more vulnerable to immune-mediated targeting and killing.

The clinical data shown above related to FIGS. 9-12 support the suggestion that rapamycin enhances the efficacy of CAR T cell therapies expressing exogenous IFN-γ. Specifically, the data shows that a patient receiving rapamycin in combination with PAP CAR T cells exhibited a more favorable PSA response compared to patients who did not receive rapamycin. This is evidenced by the stabilization of PSA levels at a normal range over an extended period for the patient treated with rapamycin, suggesting an improved therapeutic outcome. This observation implies that rapamycin could potentially modulate the tumor microenvironment or T cell functionality in a way that enhances the anti-tumor activity of CAR T cells, particularly those engineered to utilize or respond to IFN-γ signaling. Accordingly, the clinical data and the in vitro data here suggest that rapamycin can indirectly benefit T cell therapies, particularly those exploiting the IFN-γ pathway for cancer cell targeting. Rapamycin's upregulation of IFN-γ receptors and signaling components in cancer cells may increase their susceptibility to T cell mediated attack. This mechanism can enhance the efficacy of T cell therapies, especially in cases where T cells are engineered to produce or respond to IFN-γ, by making the tumor environment more amenable to immune destruction.

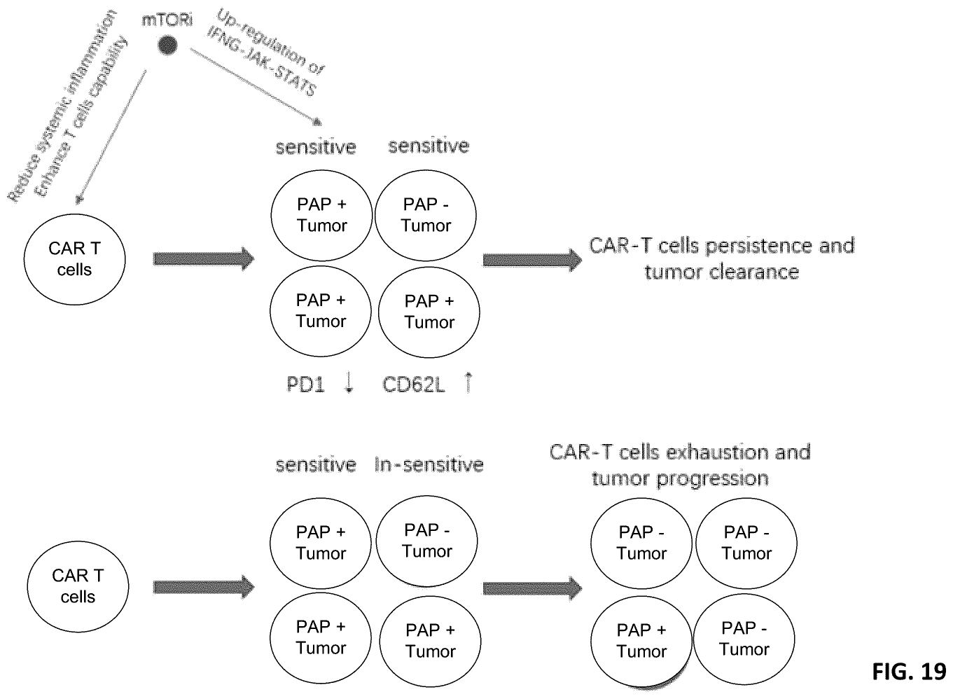

FIG. 19 summarizes the findings related to rapamycin use in cell therapies. The data are split into two parts: the lower part represents the control group, and the upper part represents the CAR T cells treated with the mTOR inhibitor, rapamycin. With rapamycin Treatment, rapamycin reduces systemic inflammation when CAR T cells are present. It enhances memory CAR T cells (CD62L), boosting their persistence. Rapamycin upregulates the IFN-γ-JAK-STAT pathway in tumor cells, increasing their sensitivity to treatment. Both positive (PAP+) and negative (PAP-) tumor cells become more sensitive to CAR T cell therapy. CAR T cells downregulate PD1 expression and upregulate CD62L, shifting towards a memory phenotype that is more persistent and effective in tumor clearance.

Without rapamycin treatment, CAR T cells show weaker sensitivity to PAP tumor cells. CAR T cell functionality is not enhanced in the absence of rapamycin. The figure shows that rapamycin has a dual effect, making tumor cells more susceptible to immune attack. It improves CAR T cell functionality by promoting a memory phenotype essential for long-term antitumor activity. Overall, rapamycin is a beneficial adjunct in CAR T cell therapy by modulating both the tumor cells and T cells to enhance the antitumor immune response.

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method of enhancing anti-tumor activities of modified cells, the method comprising:
administering an effective amount of the modified cells to a subject having a solid tumor; and
administering an effective amount of an agent to the subject, the agent comprising rapamycin;
wherein:
the modified cells inhibit growth of the solid tumor in the subject,
the anti-tumor activities in the subject are greater than those in a subject that is administered with an effective amount of modified cells but without the agent, and
the modified cells comprise T cells or NK cells, or a combination thereof.

2. The method of claim 1, wherein the modified cells comprise an exogenous nucleotide acid encoding IFN-γ.

3. The method of claim 2, wherein enhancing the anti-tumor activities of the modified cells comprises upregulating IFN-γ sensitivity of the solid tumor.

4. The method of claim 1, wherein the modified cells comprise a chimeric antigen receptor (CAR) binding a solid tumor antigen.

5. The method of claim 4, wherein the solid tumor antigen comprises tumor associated MUC1 (tMUC1), PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, B7-H3, MAGE A4, EGFR, or a combination thereof.

6. The method of claim 4, wherein the solid tumor antigen comprises tMUC1, ACPP, TSHR, GUCY2C, UPK2, MAGE A4, CLDN18.2, or a combination thereof.

7. The method of claim 4, wherein the CAR comprises an antigen binding domain, a transmembrane domain, a co-stimulatory domain, and a CD3 zeta domain.

8. The method of claim 7, wherein the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof; and/or wherein the first CAR comprises a scFv binding CD19, and an intracellular domain of 4-1BB or CD28, and CD3 zeta domain, and the second CAR comprises a scFv binding tMUC1, ACPP, TSHR, GUCY2C, or CLDN18.2, and an intracellular domain of 4-1BB or CD28, and CD3 zeta domain.

9. The method of claim 1, wherein the modified cells comprise a first population of cells comprising a first CAR binding a first antigen, and a second population of cells comprising a second CAR binding a second antigen, and wherein the second antigen is different from the first antigen.

10. The method of claim 9, wherein the first antigen comprises a cell surface molecule of a white blood cell (WBC).

11. The method of claim 10, wherein the WBC comprises a granulocyte, a monocyte, a lymphocyte, or a combination thereof.

12. The method of claim 10, wherein the WBC is a B cell.

13. The method of claim 10, wherein the cell surface molecule of the WBC comprises CD19, CD22, CD20, BCMA, CD5, CD7, CD2, CD16, CD56, CD30, CD14, CD68, CD11b, CD18, CD169, CD1c, CD33, CD38, CD138, CD13, or a combination thereof.

14. The method of claim 1, wherein the modified cells comprise a vector encoding at least one or more of IL-6, IL-12, IL-15, IL-7, TNF-α, or IFN-γ.

15. The method of claim 1, wherein the modified cells comprise T cells comprising a modified TCR.

16. The method of claim 1, wherein administering an effective amount of the agent to the subject comprises administering an effective amount of rapamycin to the subject at a dose of about 0.6-60 mg per subject.

17. The method of claim 1, wherein administering an effective amount of the agent to the subject comprises administering an effective amount of rapamycin to the subject at a dose of about 1-16 mg per subject.

18. The method of claim 1, wherein administering an effective amount of the agent to the subject comprises administering an effective amount of rapamycin to the subject at a dose of about 1-10 mg per subject.

19. The method of claim 1, wherein administering an effective amount of the agent to the subject comprises administering an effective amount of rapamycin to the subject at a dose of about 1-6 mg per subject.

20. The method of claim 1, wherein administering an effective amount of the agent to the subject comprises administering an effective amount of rapamycin to the subject more than 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days after the subject has been administered the effective amount of the modified cells.

21. The method of claim 1, wherein administering an effective amount of the agent to the subject comprises administering an effective amount of rapamycin to the subject at about when CAR T cell number reaches a peak after the subject has been administered the effective amount of the modified cells.

22. A method of enhancing anti-tumor activities of CAR T cells, the method comprising:
administering an effective amount of the CAR T cells to a subject having a solid tumor, the CAR T cells comprising exogenous nucleotide acid encoding IFN-γ; and
administering an effective amount of an agent to the subject, the agent comprising rapamycin; wherein the anti-tumor activities in the subject are greater than those in a subject that is administered with an effective amount of CAR T cells but without the agent, and wherein enhancing the anti-tumor activities of the modified cells comprises enhancing the solid tumor's responsiveness to IFN-γ signaling.

23. The method of claim 1, further comprising at least one of:
monitoring concentration of the CAR T cells in the blood of the subject;
determining one or more days corresponding to one or more peaks of the concentration of CAR T cells in the blood;
administering the effective amount of the agent at around the one or more days, and
measuring the anti-tumor activities 20, 30, 40, 50, 60, 70, 80, 90, or 120 days after the subject has been administered the effective amount of the modified cells, wherein the anti-tumor activities in the subject are greater than those in a subject that is administered with an effective amount of modified cells but without the agent 20, 30, 40, 50, 60, 70, 80, 90, or 120 days after the subject has been administered the effective amount of the modified cells, indicates enhanced anti-tumor activities of the modified cells.
